(19) United States
(12) Patent Application Publication
(10) Pub. No.: US 2026/0305977 A1
(43) Pub. Date: Oct. 8, 2026

(54) METHODS, SYSTEMS AND DEVICES FOR AUTOMATED LIMB-BASED WEIGHT ESTIMATION

(71) Applicant: Nura Medical Inc., Montreal (CA)

(72) Inventors: Jean-Gabriel LACOMBE, Montreal (CA); Georgia POWELL, Montreal (CA); Kevin ARMOUR, Montreal (CA); Dylan HORVATH, Toronto (CA); Eugenie DE LOYNES, Toronto (CA); Parth JAIN, Toronto (CA); Andrew LOWE, Toronto (CA)

(73) Assignee: Nura Medical Inc., Montreal, QC (CA)

(21) Appl. No.: 19/483,113

(22) PCT Filed: May 15, 2024
(86) PCT No.: PCT/CA2024/050649

§ 371 Date: Nov. 10, 2025

Related U.S. Application Data
(60) Provisional application No. 63/466,387, filed on May 15, 2023.

(51) Int. Cl. A61B 5/107; A61B 5/00

(52) U.S. Cl. CPC A61B 5/1072; A61B 5/4839; A61B 5/6831

(57) ABSTRACT

Described herein are methods, systems and devices for automated limb-based weight estimation for use in, for example, automated weight-based medication dose preparation systems and methods. In at least one embodiment, the system comprises a weight estimation device comprising a disposable strip configured to be applied around a limb of a patient, the strip being movable between an unmounted position and a mounted position, and when in the mounted position, the strip forming a variable circumference that engages around the patient's limb. Described herein are also a number of different devices for receiving or reading information related to the variable circumference from the disposable strip.

### CROSS-REFERENCE TO PREVIOUS APPLICATION

This application claims priority from U.S. provisional patent application No. 63/466,387 filed on May 15, 2023, which is incorporated herein by reference in its entirety.

### FIELD

Various embodiments are described herein that generally relate to weight-based medication preparation, and in particular, to methods, systems and devices for automated limb-based weight estimation.

### BACKGROUND

The following is not an admission that anything discussed below is part of the prior art or part of the common general knowledge of a person skilled in the art.

In clinical settings (i.e., hospital and emergency rooms), health practitioners are often required to administer weight-based medication dosages (i.e., boluses) to patients. To this end, prior to administering the medication dosage, health practitioners will typically complete a weight assessment of the patient, which is then used to perform a weight-based dosage calculation.

It has, however, been appreciated that the timely and accurate calculation, preparation, and administration of weight-based medications presents a major challenge for healthcare professionals, especially in the provision of care for pediatric patients.

In particular, it has been appreciated that errors generally occur at three main points in the process of preparing weight-based medication: (a) estimation of the patient's weight, (b) calculation of the correct volumes of medication and/or diluent, and (c) preparation of the bolus dose, i.e., the syringes and/or or intravenous (IV) bags. Together, these sources of error present a barrier to the delivery of appropriate care and can lead to severe adverse effects for patients.

Additionally, the tools used in the assessment of weight-based medication preparation may require additional sanitization and/or cleaning between uses. These procedures may impede the work of healthcare professionals in treating their patients.

### SUMMARY OF VARIOUS EMBODIMENTS

Generally, the present disclosure is directed to methods, systems and devices for automated limb-based weight estimation.

In accordance with one aspect of the present disclosures, there is provided a weight estimation device comprising a disposable strip configured to be applied around a limb of a patient, the strip having a first portion and a second portion, the first portion including a plurality of indicators and the second portion being configured to receive an encoder module for monitoring the position of the first portion of the strip with respect to the second portion of the strip using the indicators. The disposable strip is movable between an unmounted position and a mounted position and when in the mounted position, the strip engages around the patient's limb. The disposable strip is configured such that, when in the mounted position and the encoder module is coupled to the second portion, the encoder module is spaced apart from the limb of the patient.

In accordance with another aspect of the present disclosure, there is provided a weight estimation device. The wait estimation device comprises a disposable strip configured to be applied around a limb of a patient. The disposable strip is movable between an unmounted position and a mounted position, and when in the mounted position, the disposable strip forming a variable circumference that engages around the patient's limb, the disposable strip having a first portion and a second portion, the first portion including a plurality of indicators and the second portion being configured to receive an encoder module. The device also includes a tensioning mechanism having a sliding member and a resistance member, the sliding member being operable to move past the resistance member in a first direction to decrease the circumference and is resisted from moving in a second direction opposite the first direction by the resistance member.

In some examples, the plurality of indicators are at least one of optical indicators and magnetic indicators.

In some examples, the optical indicators are incrementally spaced apart ticks.

In some examples, the device further comprises a tensioning mechanism having a sliding member and a resistance member.

In some examples, the resistance member causes resistance due to friction.

In some examples, the resistance member is foam.

In some examples, the resistance member includes a ratchet system having a clip and teeth mechanism.

In some examples, the teeth are positioned on the sliding member.

In some examples, the clip is positioned on one of the disposable strip and the encoder module.

In some examples, the sliding member is positioned on the disposable strip and the resistance member is positioned on one of the disposable strip and the encoder module.

In some examples, the clip is positioned on the encoder module and is removeable from the encoder module.

In some examples, the disposable strip is configured to removably receive the encoder module in the second portion of the disposable strip.

In some examples, the disposable strip is configured to slidingly receive the encoder module in the second portion of the disposable strip.

In some examples, the disposable strip is configured to adhesively receive the encoder module in the second portion of the disposable strip.

In some examples, the first portion of the disposable strip is narrower than the second portion of the disposable strip.

In some examples, the disposable strip has a first end and a second end with a disposable strip body extending therebetween, the first end being proximate the second portion, and the disposable strip body has a semi-rigid section proximate the second end and a flexible section between the semi-rigid section and the second portion.

In accordance with another aspect of the present disclosure, there is provided a weight estimation system comprising a weight estimation device in accordance with any of the above paragraphs and an encoder module. The encode module comprises an encoder for monitoring the position of the first portion of the disposable strip with respect to the second portion; and a device processor. The device processor is configured to determine, based on data received from the encoder, a circumference of the limb and estimate a weight of the patient based on the circumference.

In some examples, the disposable strip is of one type of a plurality of different disposable strip types, each disposable strip type having a difference size and the device processor being further configured to determine the type of disposable strip and use the type of disposable strip to estimate the weight of the patient.

In some examples, the type of disposable strip is indicated by at least one of visual, magnetic, and electronic indicators.

In accordance with yet another aspect of the present disclosure, there is provided a disposable strip configured to be applied around a limb of a patient. The strip is movable between an unmounted position and a mounted position, and when in the mounted position, the disposable strip forms a variable circumference that engages around the limb. The disposable strip has a first portion and a second portion, the first portion including a plurality of indicators and the second portion being configured to receive an encoder module. The disposable strip also has a tensioning mechanism for varying the circumference, the tensioning mechanism having a sliding member and a resistance member. The sliding member is operable to move past the resistance member in a first direction to decrease the circumference and is resisted from moving in a second direction opposite the first direction by the resistance member.

In accordance with yet another aspect of the present disclosure, there is provided a disposable strip configured to be applied around a limb of a patient, the disposable strip being movable between an unmounted position and a mounted position, when in the mounted position, the disposable strip forms a variable circumference that engages around the limb. The disposable strip has a first portion and a second portion, the first portion including a plurality of indicators and the second portion being configured to receive an encoder module; and a tensioning mechanism for varying the circumference. The tensioning mechanism has a sliding member positioned on the strip that is operable to engage with a resistance member positioned on the encoder module. The sliding member is operable to move past the resistance member in a first direction to decrease the circumference and is resisted from moving in a second direction opposite the first direction by the resistance member.

In some examples, the plurality of indicators are at least one of optical indicators and magnetic indicators.

In some examples, the optical indicators are incrementally spaced apart ticks.

In some examples, the resistance member causes resistance due to friction.

In some examples, the resistance member is foam.

In some examples, the resistance member includes a ratchet system having a clip and teeth mechanism.

In some examples, the disposable strip is configured to receive the encoder module in the second portion of the disposable strip.

In some examples, the disposable strip is configured to slidingly receive the encoder module in the second portion of the disposable strip.

In some examples, the disposable strip is configured to adhesively receive the encoder module in the second portion of the disposable strip.

In some examples, the first portion of the disposable strip is narrower than the second portion of the strip.

In some examples, the disposable strip further comprises a first end and a second end with a disposable strip body extending therebetween, the first end being proximate the second portion. The disposable strip body has a semi-rigid section proximate the second end and a flexible section between the semi-rigid section and the second portion.

In accordance with yet another aspect of the present disclosure, there is provided a weight estimation system. The system comprises a disposable strip in accordance with the above and an encoder module. The encoder module comprises an encoder for monitoring the position of the first portion of the disposable strip with respect to the second portion and a device processor being configured to: determine, based on data received from the encoder, a circumference of the limb; and estimate a weight of the patient based on the circumference.

In some examples, the disposable strip is of one type of a plurality of different disposable strip types, each disposable strip type having a difference size. The device processor is further configured to: determine the type of disposable strip, and use the type of disposable strip to estimate the weight of the patient.

In some examples, the type of disposable strip is indicated by at least one of visual, magnetic, and electronic indicators.

In accordance with yet another aspect of the present disclosure, there is provided a limb circumference measuring device. The device comprises a strip configured to be applied around a patient's limb. The strip comprises a first portion that is couplable to itself such that a circumference is formed, the circumference having a variable length that is moveable from a first length to a second length; and a second portion that is operable to communicate data corresponding to the second length to a remote device. The strip is movable between an unmounted position and a mounted position, when in the mounted position the strip engages around the patient's limb such that the circumference of the first portion is applied around the patient's limb.

In some examples, the first portion is removable from the patient's limb by applying a force above a threshold force.

In some examples, the first portion has a ratchet system that allows for the variable length of the circumference to decrease from the first length to the second length with an applied force.

In some examples, the ratchet system includes a clip and teeth mechanism that prevents the variable length from increasing once the circumference has been formed.

In some examples, the clip and teeth mechanism allows for incremental decreasing of the variable length.

In some examples, the ratchet system is frangible above an applied threshold force.

In some examples, the ratchet system is manually operable to move from the first length to the second length.

In some examples, the ratchet system is automatically movable from the first length to the second length.

In some examples, the device further comprises at least one force transducer to determine a force of the first portion applied to the patient's limb.

In some examples, the second portion is operable to communicate analog data.

In some examples, the analog data communication includes a visual indicator.

In some examples, the visual indicator is at least one of a barcode, colour-coded system, and increments.

In some examples, the second portion is operable to communicate digital data.

In some examples, the second portion further comprises an RFID.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A shows an example embodiment of an environment in which the methods, systems and devices disclosed herein may operate;

FIG. 1B shows another example embodiment of an environment in which the methods, systems and devices disclosed herein may operate;

FIG. 2 is a system for automated weight-based medication dose preparation, according to some example embodiments;

FIG. 3A is an example graphical user interface (GUI) displaying an introductory, or welcome screen associated with an automated medication dose preparation software;

FIG. 3B is an example GUI associated with the automated medication dose preparation software, and displaying fields to be populated with patient-specific data;

FIG. 3C is an example GUI associated with the automated medication dose preparation software, and displaying options for medication selection;

FIG. 3D is an example GUI associated with the automated medication dose preparation software, and displaying suggested medication dosage;

FIG. 3E is an example GUI associated with the automated medication dose preparation software, and displaying suggested medication dosage parameters;

FIG. 3F is an example GUI associated with the automated medication dose preparation software, and displaying further suggested medication dosage parameters;

FIG. 4A is a front perspective view of an example embodiment of a weight estimation device;

FIG. 4B is a front perspective view of an example embodiment of a weight estimation device and docking station;

FIGS. 5A-5C are top views of a limb circumference measuring device with various lengths;

FIGS. 6A-6B are top views of a second portion of the limb circumference measuring device in various positions;

FIG. 6C is a top perspective view of the second portion of the limb circumference measuring device;

FIG. 7 is a simplified block diagram of electrical hardware components in an example weight estimation device and an example computer terminal;

FIG. 8 is a process flow for an example embodiment of a method for dose preparation;

FIG. 9 is a process flow for an example embodiment of a method for automated weight estimation;

FIG. 10A is an example plot showing the relationship between arm circumference and body weight for subjects within an age range of 0 to 4 years old;

FIG. 10B is an example plot showing the relationship between arm circumference and body weight for subjects within an age range of 5 to 7 years old;

FIG. 10C is an example plot showing the relationship between arm circumference and body weight for subjects within an age range of 8 to 19 years old;

FIG. 11 shows a perspective view of another example embodiment of a weight estimation device having an encoder module and a strip;

FIG. 12 shows a bottom perspective view of the weight estimation device of FIG. 11;

FIG. 13 shows an exploded view of the weight estimation device of FIG. 11;

FIG. 14 shows a top view of the strip of FIG. 11;

FIG. 15 shows a cross-sectional view of the encoder module and strip of FIG. 11 taken along section line 15-15 in FIG. 11;

FIG. 16 shows a perspective view of another example embodiment of a weight estimation device having an encoder module and a strip;

FIG. 17 shows a bottom perspective view of the weight estimation device of FIG. 16;

FIG. 18 shows a bottom perspective view of the encoder module of FIG. 16;

FIG. 19 shows a top view of the strip of FIG. 16;

FIG. 20 shows a cross-sectional view of the encoder module and strip of FIG. 16 taken along section line 20-20 in FIG. 16;

FIG. 21 shows a perspective view of another example embodiment of a weight estimation device having an encoder module and a strip;

FIG. 22 shows a bottom perspective view of the weight estimation device of FIG. 21;

FIG. 23 shows a bottom perspective view of the encoder module of FIG. 21;

FIG. 24 shows a top view of the strip of FIG. 21; and

FIG. 25 shows a cross-sectional view of the encoder module and strip of FIG. 21 taken along section line 25-25 in FIG. 21.

Further aspects and features of the example embodiments described herein will appear from the following description taken together with the accompanying drawings.

### DESCRIPTION OF VARIOUS EMBODIMENTS

Various systems or methods will be described below to provide an example of an embodiment of the claimed subject matter. No embodiment described below limits any claimed subject matter and any claimed subject matter may cover methods or systems that differ from those described below. The claimed subject matter is not limited to systems or methods having all of the features of any one system or method described below or to features common to multiple or all of the apparatuses or methods described below. It is possible that a system or method described below is not an embodiment that is recited in any claimed subject matter. Any subject matter disclosed in a system or method described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device. As used herein, two or more components are said to be “coupled”, or “connected” where the parts are joined or operate together either directly or indirectly (i.e., through one or more intermediate components), so long as a link occurs. As used herein and in the claims, two or more parts are said to be “directly coupled”, or “directly connected”, where the parts are joined or operate together without intervening intermediate components.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, any recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g., a pushbutton keyboard, mouse, a touchscreen, and the like), and at least one output device (e.g., a display screen, a printer, a wireless radio, and the like) depending on the nature of the device.

It should also be noted that there may be some elements that are used to implement at least part of one of the embodiments described herein that may be implemented via software that is written in a high-level computer programming language such as object-oriented programming or script-based programming. Accordingly, the program code may be written in Java, Swift/Objective-C, C, C++, Javascript, Python, SQL or any other suitable programming language and may comprise modules or classes, as is known to those skilled in object-oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage. The computer program product may also be distributed in an over-the-air or wireless manner, using a wireless data connection.

The term “software application” or “application” refers to computer-executable instructions, particularly computer-executable instructions stored in a non-transitory medium, such as a non-volatile memory, and executed by a computer processor. The computer processor, when executing the instructions, may receive inputs and transmit outputs to any of a variety of input or output devices to which it is coupled. Software applications may include mobile applications or “apps” for use on mobile devices such as smartphones and tablets or other “smart” devices.

A software application can be, for example, a monolithic software application, built in-house by the organization and possibly running on custom hardware; a set of interconnected modular subsystems running on similar or diverse hardware; a software-as-a-service application operated remotely by a third party; third party software running on outsourced infrastructure, etc. In some cases, a software application also may be less formal, or constructed in ad hoc fashion, such as a programmable spreadsheet document that has been modified to perform computations for the organization's needs.

Software applications may be deployed to and installed on a computing device on which it is to operate. Depending on the nature of the operating system and/or platform of the computing device, an application may be deployed directly to the computing device, and/or the application may be downloaded from an application marketplace. For example, user of the user device may download the application through an app store such as the Apple App Store™ or Google™ Play™.

Current methods for weight-based medical dose preparation often suffer from a number of significant drawbacks. For instance, in many cases, weight-based dose preparation may be manually performed by health care practitioners—i.e., nurses in emergency departments, or by a centralized or de-centralized pharmacy team in other hospital units. In particular, health practitioners may initially weight the subject patient, and may then perform manual calculations to determine the appropriate medication dosage. Manual determinations of this nature are, however, subject to significant human-error, and may yield inaccurate results that prove detrimental to the patient's health. In developing countries, the problems are even more acute, as medical facilities often lack scales as well as other basic tools required to accurately assess patient weight.

In recent years, there have been some attempts at automating the process of weight-based dose calculations using tools such as smartphone applications and computer software. However, many software solutions require manual input of patient data (i.e., thereby increasing likelihood of input errors), often lack in reliability and can be inaccessible to health staff. Applications and software solutions also do not offer case-dependent preparation support, and do not assist in the prioritization of medications or identification of potential adverse drug interactions. Additionally, current automated IV medication preparation software systems focus primarily on oncology or on large-scale pharmaceutical use.

It has been further appreciated that, in the special context of pediatrics-current available tools utilize recumbent length and/or age of the child as a predictor of weight. These approaches have been shown to perform well only in specific subsets of the population and do not address body composition and ethnic differences. Moreover, many of the methods utilizing length are not appropriate for tall children and young teenagers. Still further, many current solutions do not allow for real-time or near real-time monitoring of pediatric patients' weight. This has particular significance in pediatric ICUs, where all medication is weight-based-accordingly, having an updated and accurate weight is critical for providing pediatric patients with the correct medication dose.

Still further yet, the inventors have appreciated that there are no existing effective tools which can, in addition to automatically determining appropriate weight-based bolus dosage, can also automatically prepare the bolus dose, i.e., automatically prepare a syringe or IV bag. If present, such tools would have significance in eliminating, or reducing, errors in the bolus preparation process.

In view of the foregoing, to mitigate at least some of the aforementioned drawbacks of conventional methods, embodiments herein provide for methods, systems and devices for automated limb-based weight estimation.

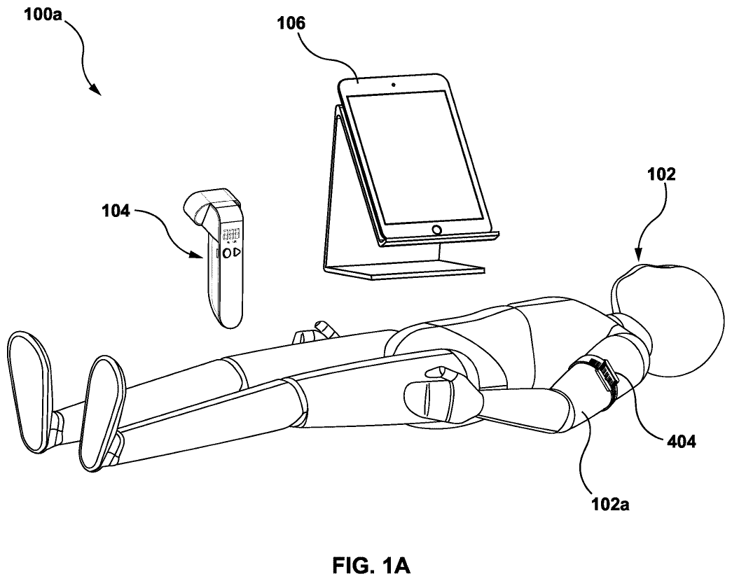

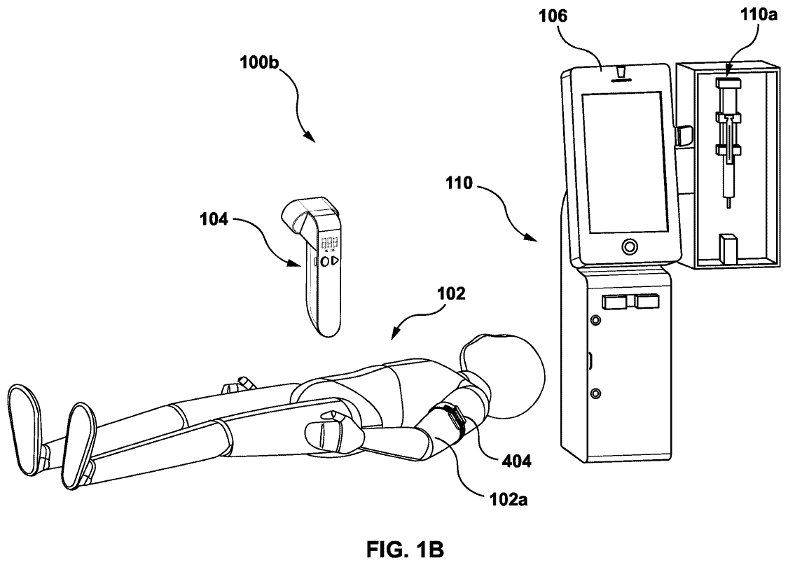

Reference is now made to FIG. 1A, which shows an example environment 100a in which the methods and systems disclosed herein may operate, in accordance with some embodiments.

As shown, a patient 102 may be located in a medical or clinical setting, and may require delivery of a weight-based dose medication, i.e., orally, via a syringe, IV line or interosseous infusion, etc. Patient 102 may be, for example, a pediatric patient.

To mitigate for at least some of the aforementioned challenges in accurate estimation of the patient's weight, embodiments herein provide for a limb-based weight estimation device 104. The limb-based weight estimation device 104 can be used for real-time or near real-time weight estimation. In some embodiments, the limb-based weight estimation device 104 may be a part of a weight estimation system 900. The weight estimation system 900 may include a limb circumference measuring device 404 and a weight estimation device 104 that is separate from the limb circumference measuring device 404. As exemplified, the weight estimation device may be a scanning device and the limb circumference measuring device 404 may be a strip.

As shown, the strip 404 may be applied around the subject's limb to measure the limb circumference of the patient. As provided herein, the measured limb circumference is used as a surrogate to estimate the patient's weight. In the illustrated embodiment, the strip 404 is applied around the subject's arm 102a to measure the middle-upper arm circumference. In other cases, however, the strip 404 may also be applied around the subject's thigh to measure thigh circumference, around the subject's calf to measure calf circumference or otherwise any other suitable part of the arm or leg. In any case, the strip 404 can be applied to the patient, irrespective of the position of the patient (i.e., standing, sitting or lying down). This may be particularly advantageous for patients, i.e., pediatric patients, who are immobile.

In at least some embodiments, the device 104 may be in communication with and/or receive information from the strip 404 to automatically determine the subject's weight based on the measured limb circumference using a circumference-based weight prediction algorithm. The estimated weight data is then communicated to an external computing device 106 to determine weight-based medication dosage for a given medication type. In other embodiments, device 104 may determine and transmit the patient limb circumference data, i.e., to the external computing device 106. The external computing device 106 may then estimate subject's weight.

To this end, the concept of using limb circumference as an estimate of weight is premised on the notion that surrogate limb circumference measurements generally introduce less error in weight prediction than existing methods that rely on other factors, such as recumbent length and/or subject age. The use of limb circumference also significantly extends the age range for which weight estimates can be obtained through use of surrogate measurement. Additionally, the use of the automated device 104 increases repeatability and reliability of measured surrogate measurements, and simplifies the measurement process. The automated weight estimation system may significantly reduce rate of human error from user-dependent weight estimates. As the weight estimation data is automatically communicated to computing terminal 106, this can also further reduce the risk of human error by removing the need for manual input of patient data into applications and/or software systems operating on terminal 106.

External computing terminal 106 may host a medication dose preparation software. The medication dose software is operable to perform weight-to-medication computation. The weight-to-medication computation converts, weight estimation data—i.e., received from the system 900, as well as other patient-specific data (i.e., gender, age, etc.), to correct medication therapeutic dosage ranges for a given medication. In various cases, this conversion is performed using pre-defined medication libraries, which provide optimal, or standard, medication dosage (mg/kg) for different patient weights and for different medication types.

In some embodiments, as stated previously, computing terminal 106 may not receive weight estimation data from device 104, but may only receive limb circumference data. In other words, rather than estimating weight—the weight estimation device 104 may only transmit limb circumference data to terminal 106. In these cases, prior to performing weight-to-medication computation, the software may initially estimate patient weight from received limb circumference data.

In at least one embodiment, the medication dose software—operating on computing terminal 106—may present various procedural information for preparing and administering medication. In some cases, where multiple medication types are administrated to the patient, the medication dose software can also enable automatic prioritization of administered medications according to standard of care algorithms. The software may also detect pharmaceutical interactions between multiple medication types, and may warn the health practitioner of adverse effects. This can be performed using pre-defined clinical treatment flows and/or via aggregation of data and statistical predictions according to historical treatment flows. In general, the collective of these features reduces the need for manual calculations and provide further insight to healthcare professionals on the preparation of time-sensitive medications. In some embodiments, other features could include a timer for resuscitation medications that need to be administered at specific time intervals, a timer for chest compressions and/or report generation for transfer of care.

While the illustrated embodiment has shown the computing terminal 106 and weight estimation device 104 as being separate components—it will be understood that in other embodiments, these may in-fact be a single integrated component. For example, the weight estimation device 104 may include integrated computing functionality for hosting the medication dose software.

In view of the foregoing discussion, it will now be appreciated that the weight estimation device 104 and the medication dose software—operating on computing terminal 106—taken alone or in combination, have a number of useful applications.

In one example application, the combination of the weight estimation device 104 and medication dose software may be applied in a clinical setting, such as an emergency room. For instance, a pediatric patient having an unknown weight, and in critical condition, may be presented to an emergency department. Upon presentation, healthcare practitioners may immediately apply the strip 404 on the patient to communicate with the weight estimation device 104, and may use the medication dose software—on computer terminal 106—to determine appropriate dosage parameters (e.g., suggested dosage, total dosage, drug volume, etc.) for one or more medications prescribed by an attending physician. In addition to determining dosage, the medication dose software may also provide guiding instructions for preparing and administering the medication, as well as providing guidance on the prioritization order of multiple administered medication doses. Accordingly, in this example case, the concurrent use of the weight estimation device 104 and medication dose software provides for accurate determination and administration of medication doses in a time-sensitive manner.

In another example application, the weight estimation device 104 and medication dose software may be used in intensive care units (ICU). In ICUs, obtaining a patient's accurate weight can present a challenge as the majority of patients are immobile and are connected to various medical equipment, such as IV lines and oxygen masks. The weight estimation device 104 accordingly provides a convenient solution for measuring weight of patients in an immobile state. Additionally, the weight estimation device 104 may have practical benefit for real-time or near real-time monitoring of a pediatric patient's weight, or otherwise any patient's weight. In particular, in pediatric ICUs, all medications are weight-based, therefore having an updated and accurate weight is critical for providing pediatric patients with the correct medication dose. Currently, based on patient and hospital, patient weight may only be measured on an intermittent basis, i.e., weekly basis.

In yet another example application, the weight estimation device 104 and medication dose software can be used remotely from one other. For example, the weight estimation device 104 may be available in an ambulance, while the medication dose software may be operating on a computer terminal 106 located at a receiving hospital. Typically, paramedics and various emergency response teams have limited scope in their ability to perform life-saving measures. Accordingly, as the patient is being transported to the hospital, it is vital that nurses and physicians (i.e., at the receiving hospital) have advanced opportunity to prepare for the incoming trauma. In these circumstances, as the patient is on route to the hospital, the paramedics can use the weight estimation device 104, and may contact healthcare professionals in the emergency room to relay weight estimation data displayed on the device 104. In turn, health care professionals can begin advanced preparation of the weight-based medication, i.e., using the medication dose software operating on computer terminal 106, in anticipation of the patient's arrival. In this manner, the medication is readily prepared well in advance of the patient's arrival at the hospital. In other cases, rather than the paramedic verbally communicating weight estimation data to the healthcare team at the hospital-weight estimation data may also be automatically communicated by the weight estimation device 104 to the computing terminal 106 located at the hospital (i.e., via satellite or internet networks).

In some example applications, the weight estimation device 104, and the medication dose software—operating on the computing terminal 106—may be used independently.

For instance, during triage in an emergency department, the strip 404 may be applied independently to the patient upon arrival (i.e., a pediatric patient). A triage nurse may begin by assessing the patient's weight using the weight estimation device 104. The nurse may remove the strip 404 from the patient's arm after the device 104 has communicated with the strip 404, and the weight will remain on the screen of the device 104. The nurse may then record the weight in the hospital's electronic medical records system or on a paper chart and can continue their assessment of the patient.

In another example, the medication dose software may also be used independently from the weight estimation device 104, as for example in non-urgent or non-critical clinical settings. For instance, if the patient's weight is already known, a health care professional may enter the patient's weight directly into the medication dose software, without the use of the weight estimation device 104. The medication dose software may then determine the appropriate medication dose for a given medication, as well as provide guiding instructions for administering the dose.

Reference is now made to FIG. 1B, which shows another example environment 100b in which the methods and systems disclosed herein may operate, according to some other embodiments.

The environment 100b is generally analogous to the environment 100a, with the exception that the computing terminal 106 is now coupled to a bolus dose preparation system 110. Bolus dose preparation system 110 is operable to automatically prepare a bolus dose based on dosage parameters determined, for example, by the medication dose software. In various cases, the medication dose software (or some other software) may be operable to control hardware components of the bolus dose preparation system 110 to automatically fill a syringe 110a or an IV bag. This, in turn, eliminates or reduces errors associated with the actual bolus dose preparation process. In some cases, a separate computing device—which may be in communication with computing terminal 106—may control the dose preparation system 110. As will be appreciated by the skilled reader, the presently disclosed methods, systems and devices for automated limb-based weight estimation need not comprise or be coupled to a dose preparation system 110.

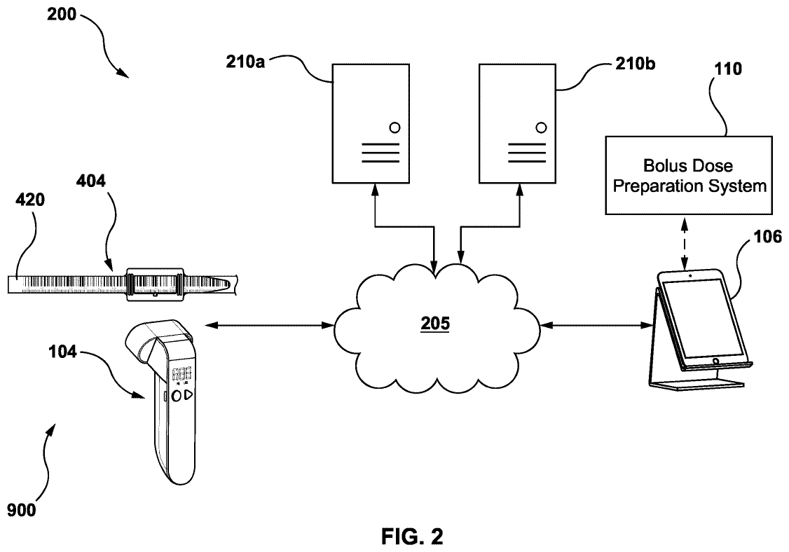

Reference is now made to FIG. 2, there is shown a system 200 for automated weight-based medication dose preparation, in accordance with some embodiments.

As shown, system 200 generally includes the system 900, including the strip 404 and the device 104, connected, via network 205, to external computing terminal 106. In some embodiments, external computing terminal 106 may itself be connected to the bolus dose preparation system 110, as explained with reference to FIG. 1B. In some cases, system 200 further includes one or more servers 210 connected to the network 205.

Network 205 may be any network capable of carrying data include any wired or wireless network. By way of non-limiting examples, network 205 may comprise an Internet network, Ethernet network, plain old telephone service (POTS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signaling network, fixed line, local area network, wide area network, and others, including any combination of these. In some cases, network 205 may be constructed from one or more computer network technologies, such as IEEE 802.3 (Ethernet), IEEE 802.11 and similar technologies. In other cases, network 205 may comprise a personal area network (PAN), such as a Bluetooth® connection. In some cases, network 205 may comprise a combination of multiple network types. For example, the communication between device 104 and computer 106 may occur over Bluetooth® connection, while the communication between device 104/computer 106 and servers 210 may occur over a non-Bluetooth® connection.

External computer terminal 106 may be any suitable computing device capable of executing software programs and applications. For example, computer terminal 106 may be a tablet computer, a smartphone, desktop or laptop computer, as well as a wide variety of “smart” devices capable of data communication. Computer terminal 106 may be portable, and may at times be connected to network 205 or a portion thereof.

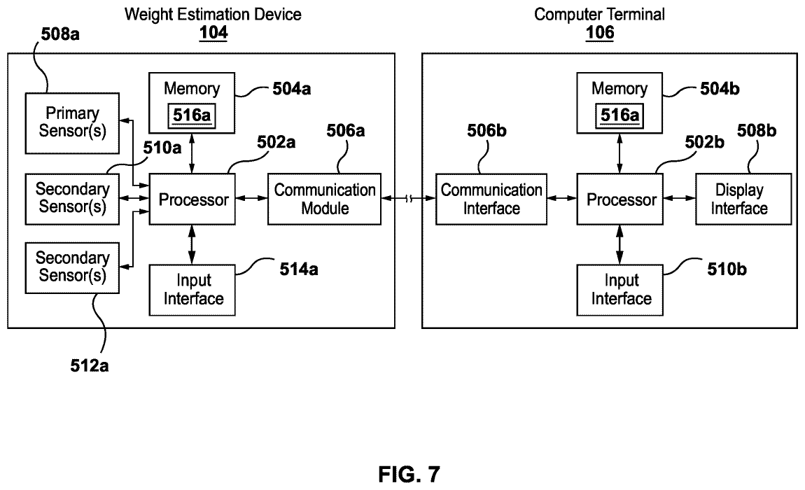

As provided in greater detail herein, with reference to FIG. 7, computer terminal 106 may include a processor 502b coupled, via a data bus, to a volatile and non-volatile memory 504b, at least one communication interface 506b, a display interface 508b and an input interface 510b.

In at least one embodiment, memory 504b—of computing terminal 106—may store the medication dose preparation software 512b. As explained previously, among other functionalities, the medication dose preparation software 512b may be operable to automatically determine bolus doses using estimated patient weight data (as well as other patient-specific data). Medication dose preparation software 512b may also detect various pharmaceutical interactions to generate warnings of adverse effects. In still other cases, medication dose preparation software 512b may be operable to control the medication dose preparation system 110.

Servers 210 are computer servers that are connected to network 205. Similar to computing terminal 106, servers 210 may also have a processor, volatile and non-volatile memory, at least one network interface, and may have various other input/output devices.

As with all devices shown in the system 200, there may be multiple servers 210, although not all are shown. It will be understood that the server 210 need not be a dedicated physical computer. For example, in various embodiments, the various logical components that are shown as being provided on server 210 may be hosted by a third party “cloud” hosting service such as Amazon™ Web Services™ Elastic Compute Cloud (Amazon EC2).

In at least one embodiment, servers 210 can include an electronic health records server 210a and/or a database server 210b. Electronic health records server 210a may include various databases for storing patient-specific data (i.e., patient IDs, gender, etc.) In some cases, electronic health records server 210a can be associated, for example, with a third-party, such as a local health service department.

Database server 210b can be used to store various databases for access by the medication preparation software 512b. For example, the database server may store pre-defined medication libraries that can be accessed to compute weight-based medication dosages. Database server 210b can also store pre-defined medication interaction data that can be used to detect pharmaceutical interactions. In other cases, this data may be stored locally on the memory 504b of the computer terminal 106.

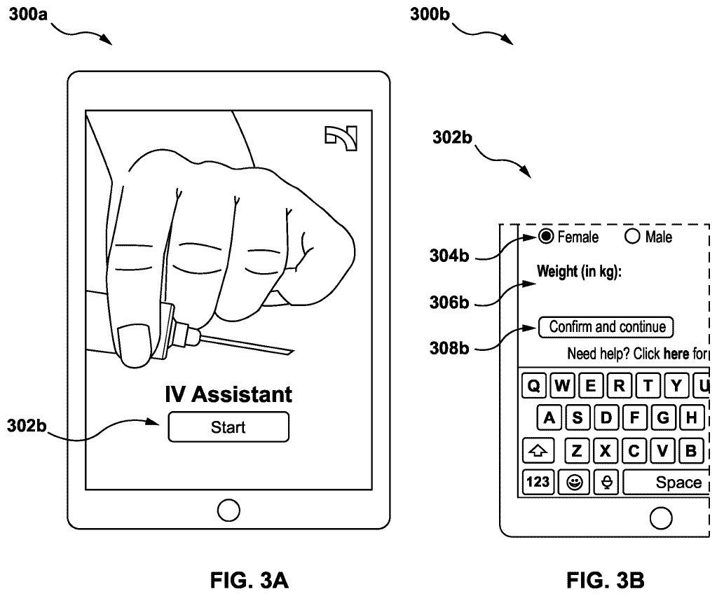

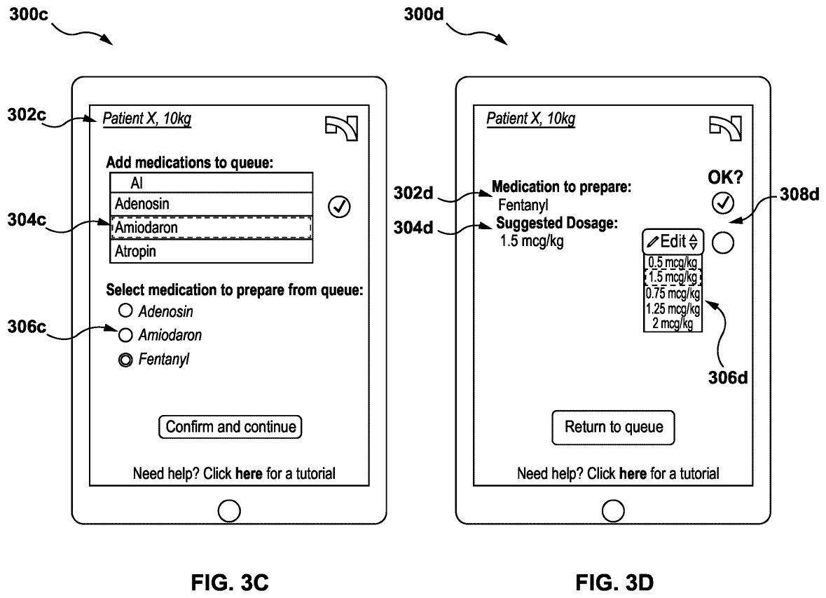

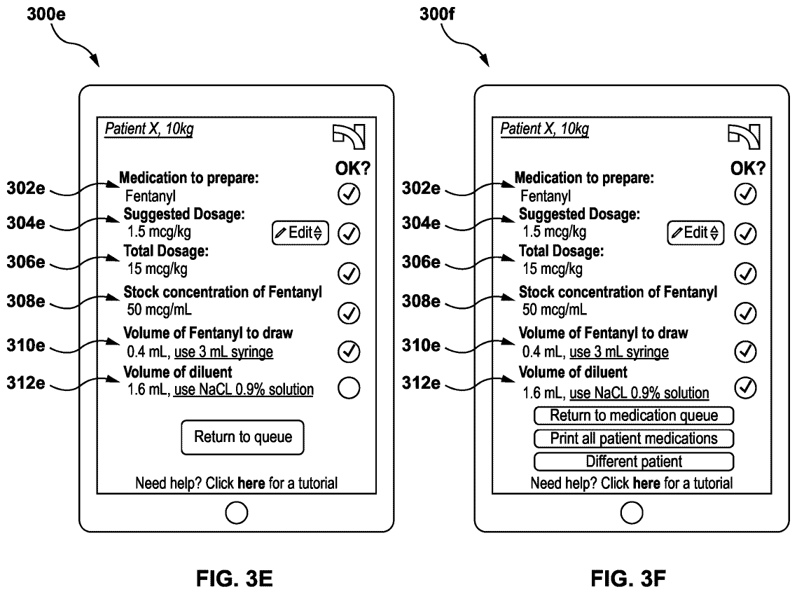

Reference is now made to FIGS. 3A-3F, which show various screenshots of example graphical user interfaces (GUIs) that may be displayed on the computer terminal 106. The GUIs may be displayed, for example, on the computer's display interface 508b, and may be generated by the bolus dose preparation software 512b executing on processor 502a (FIG. 7).

FIG. 3A illustrates an example screenshot of a GUI 300a which displays an introductory, or welcome screen. The introductory screen enables a user (i.e., a health practitioner) to select a “start” option 302a to commence medication dose preparation.

FIG. 3B illustrates an example screenshot of a GUI 300b that may follow the introductory screen 300a. Screen 300b prompts the user to enter patient-specific data. For example, the patient-specific data can include a patient ID 302b, the patient's sex 304b as well as the patient's weight 306b.

In some embodiments, rather than inputting patient-specific data, the software 506 may request—or automatically receive—the data from the electronic health records server 210a. For example, the user may enter the patient ID, and the software 506 may use the patient ID to retrieve associated patient-specific data that is pre-stored on the server 210a.

In other cases, once the user has entered the patient-specific data into the screen 300b, the software 506 may automatically communicate this data to the server 210a for recording. In some cases, this communication may occur in real-time, or near real-time, to ensure patient records on server 210a are up-to-date. In any case, once patient-specific data is entered or retrieved, the user may confirm by selecting option 308b.

FIG. 3C illustrates an example screenshot of a further GUI 300c. The GUI screen 300c may display the estimated patient weight 302c (i.e., 10 Kg). The estimated patient weight can be determined based on data received from the weight estimation device 104. For example, before, after or concurrently while the patient-specific data is being entered (or otherwise being externally received from server 210a)—the strip 404 may be applied to the patient 102. The device 104 may then be used to communicate with the strip 404 and automatically determine estimated patient weight based on measured limb circumference. Device 104 may then transmit (automatically or upon request) the estimated weight to the computer terminal 106.

In other cases, the device 104 may not estimate patient weight, and may only determine limb circumference data. In these cases, only limb circumference data is transmitted from the device 104 to the software 512b. Software 512b may then, itself, apply a circumference-based weight prediction algorithm to estimate patient weight.

As shown, screen 300c may also display an option feature 304c to enable selection between multiple types of medication to queue for administering to a patient. Option 306c enables the user to select one of the queued medications to administer to the patient.

FIG. 3D illustrates an example screenshot of a GUI 300d in which software 512b may automatically determine a suggested bolus dosage 302d for the selected medication 304d (i.e., 1 mcg/kg of Fentanyl). In embodiments provided herein, this may be determined based on analyzing the patient's estimated weight 302c, as well as the patient-specific data.

In some cases, software 512b may have access to pre-defined medication libraries that can be used to determine medication dosage based on patient weight. The medication libraries may be stored directly on the computer terminal 106, or may be accessed (or retrieved) from external server 210b. In some cases, the user may also have the option to edit or revise the dosage at 306d.

FIG. 3E shows an example screen 300e in which the software 512b may present the user with summary details in respect of the medication to prepare (i.e., Fentanyl) 302e, the suggested dosage (1.5 mcg/kg) 304e, total dosage (15 mcg) 306e, stock concentration 308e, volume of medication to draw 310e and volume of diluent 312e. In various cases, total dosage (mg) 306e may be determined as the dosage (mg/kg)×patient weight (kg). Further, the volume to draw from stock medication vial 310e may be determined by total dosage (mg)/medication stock concentration (mg/mL). In FIGS. 3E and 3F, the user may have the option to confirm each of these selection parameters.

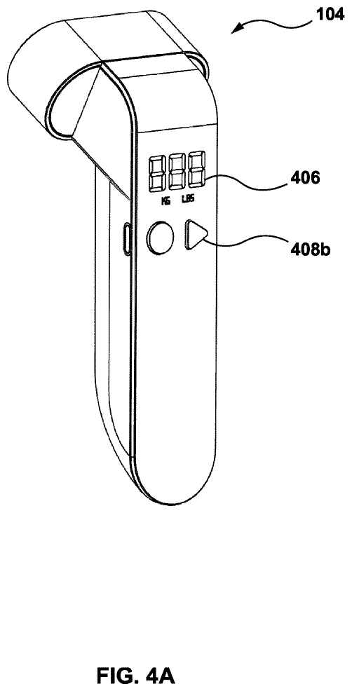

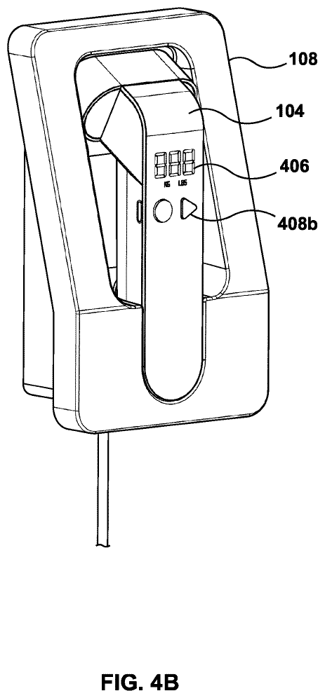

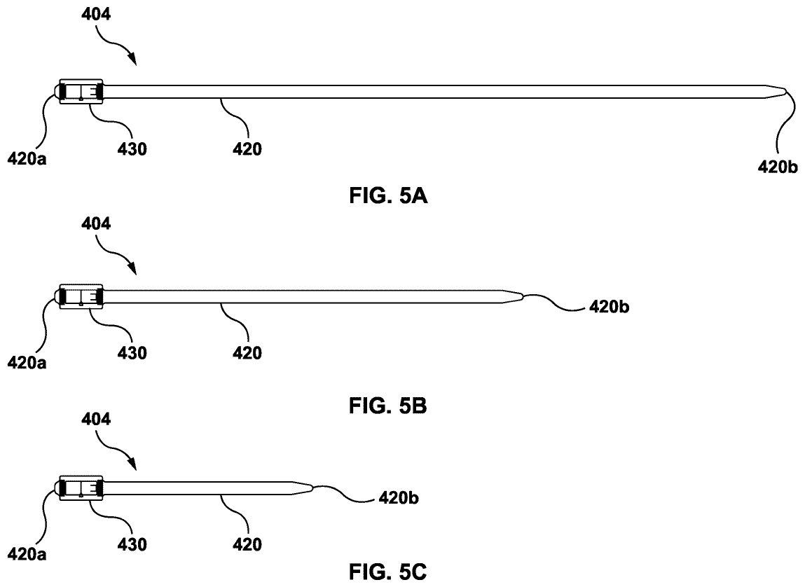

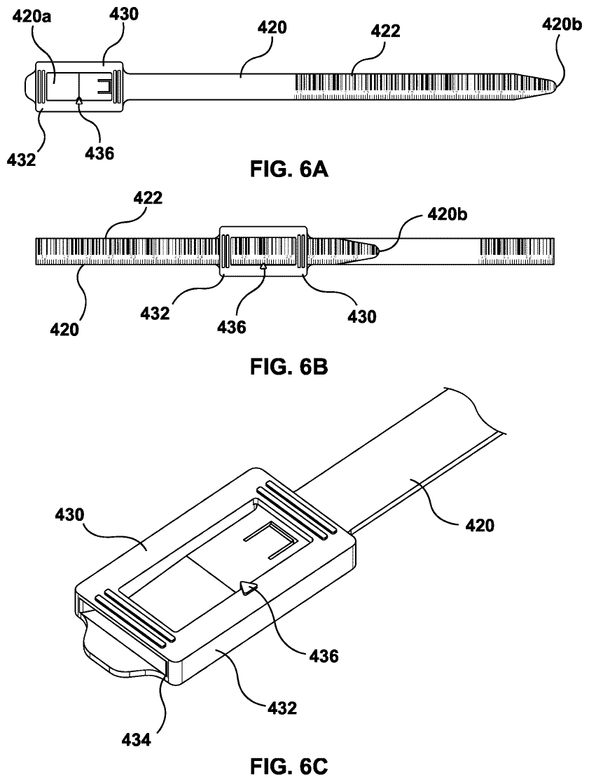

Reference is now made concurrently to FIGS. 4A-4B, which illustrate various schematic views of the weight estimation device 104, and to FIGS. 5A-6C, which illustrate various schematic views of the strip 404, according to some embodiments.

As shown, in at least some example embodiments, the device 104 can include an outer casing, which can house various device electronics. In at least some embodiments, the outer casing may be manufactured from a washable and/or durable material to enable re-use. In some cases, the material can be any one, or any combination, of plastic, metal, and synthetic materials.

In a mounted or applied position, the strip 404 is wrapped around the patient's limb (i.e., 102a in FIG. 1), i.e., in a fitted engagement around the limb, to measure a limb circumference. In an unmounted position, the strip 404 may not otherwise be in fitted engagement around the patient's limb, i.e., the strip 404 may be loose around the limb.

The strip 404 may be made of any material that is safe to contact a patient. The strip 404 may be single use (i.e., disposable) or may be cleaned and reused. The strip 404 may be removable from the limb of a patient by applying a force above a threshold force to break and/or tear the strip 404. This frangibility may allow for expedited medical care by allowing a user to quickly remove the measuring strip from the limb of the user. In at least one embodiment, the strip 404 can be manufactured of a flexible material to facilitate circumference measurements. By way of non-limiting examples, the strip 404 can be manufactured of plastic, metal, paper, linen, natural or other manmade materials. In some cases, numbers and/or linear markings are printed on strip 404 to allow for rapid visual and sensor verification of digital measurements.

The strip 404 may be movable between an unmounted position (see e.g., FIGS. 5A-5C) and a mounted position (see e.g., FIGS. 1A-1B). When in the mounted position, the strip 404 engages the patient's limb such that the circumference of the strip 404 is applied the patient's limb.

In the illustrated embodiment, the device 104 may have one or more input and output interfaces, including a device display 406 and a measure button 408b. In one example case, once the strip 404 is securely wrapped around patient's limb, the device 101 may be used to automatically determine the patient's estimated weight. The estimated weight may then be displayed on the display 406. Alternatively, or in addition, the display 406 can display the measured circumference.

The device 104 may include an encoder 412. encoder 412 can measure or track the limb circumference based on indicators 422 on strip 404. In some embodiments, the encoder is a rotary encoder. The encoder can be, by way of non-limiting examples, magnetic, optical, mechanical, or capacitive in nature.

In other cases, a linear encoder may be provided to measure limb circumference. The linear encoder can be, by way of non-limiting examples, optical, magnetic, inductive or capacitive in nature. In some cases, a combination of a linear and rotary encoder can be provided. For example, the linear encoder can provide absolute analog or digital data on large distance increments such as 10 cm. Conversely, finer, absolute or incremental length measurements can be assessed by a rotatory encoder. In an alternate embodiment, the encoding unit comprises a combination of several rotary encoders only. Similarly, another embodiment would comprise linear encoders and linear markings on the strip only. In various cases, multiple encoders can be used for redundancy and increasing measurement accuracy.

While not illustrated, the device electronics can include one or more sensors, such as pressure, force, or tactile sensors used to ensure constant pressure between measurements. In some cases, the sensor data (i.e., pressure data) may be displayed as well on the display interface 406. Additionally, a computing unit may be provided which can include a processor, memory, and/or communication interface.

In some embodiments, the digital weight estimation device provides additional patient data. Data such as blood pressure measurements, brachial pulse, bone density, body temperature, and oxygen saturation can also be provided. Similarly, additional features such as a cardiopulmonary resuscitation timer can be integrated in the weight-estimation device.

Reference is made again to FIG. 2, which shows the example system 200 for weight-based medication dose preparation including the weight estimation system 900 having the strip 404 and the weight estimation device 104.

As shown, the strip 404 has a first portion 420 and a second portion 430. The first portion 420 has a first end 420a and a second end 420b. The second portion 430 has a housing 432 with an opening 434 proximate the first end 420a of the first portion.

The first portion 420 may be moveable to form a circumference by positioning the second end 420b through the opening 434 in the housing 432 of the second portion 430. The circumference formed by the first portion 420 has a variable length that is moveable from a first length to a second length. The first length may be the initial circumference length created when the first portion 420 is threaded through the second portion 430. The first length may be larger than the second length such that the strip 404 is positionable around the limb of the patient. Alternately, the first length may be formed directly around the limb of the patient. The first portion 420 may then be tightened until the strip 404 is engaged with the limb of the patient. The second length may be referred to as the mounted position whereby the strip 404 is mounted to the limb of the patient.

Reference is now made to FIGS. 6A-6C, which illustrate various schematic views of the strip 404, according to some embodiments. The second portion 430 is operable to communicate data corresponding to the second length to a remote device. Accordingly, when the strip 404 is in the mounted position, the second portion 430 provides an indicator as to the limb circumference of the patient. The limb circumference data may subsequently be used to estimate the weight of the patient.

As shown, the first portion 420 includes an indicator 422 and the second portion 430 includes an indicator reader 436. When in the mounted position, the strip 404 may be tightened to the second length to provide the limb circumference of the user. Referring to FIG. 6B, as shown, the strip 404 is at the second length in the mounted position. The indicator reader 436 is positioned with reference to a specific indicator 422 on the first portion 420. Accordingly, the second portion 430 is in a position where it is operable to communicate data corresponding to the second length and the circumference of the limb of the patient based on the position of the indicator marker 436 relative to the indicator 422.

The indicator 422 and indicator marker 436 may be any system capable of identifying the circumference length of the mounted strip 404. The information conveyance may be analog and/or digital.

In some embodiments, as exemplified in FIGS. 6A-6B, the strip 404 is fitted with a barcode along the first portion 420 to enable retrieving of circumference data via scanning. The barcode may be used to indicate length measurements of, for example, 1 mm increments. The barcode may be, but is not limited to, linear (1D) or matrix (2D).

The analog indicators may include, for example, imaging and image capture technology. The strip 404 may feature a reference frame that is used to both guide the user and enhance image capture accuracy. For example, imaging technology may be trained to recognize select images within the specific frame.

In at least some of the embodiments, the information conveyance may be digital. For example, a radio-frequency identification (RFID) system may be used. For example, the indicators 422 may each form part of a broken circuit. When in the mounted position, the indicator marker 436 may complete a specific circuit by connecting with a particular indicator 422, indicating the limb circumference value that may be read by an RFID scanner.

It will be appreciated that the strip 404 may be any size. The strip 404 may come in several distinct sizes to accommodate limb size and/or age of the patient. For example, referring to FIGS. 5A-5C, three sizes of strip 404 are shown. These sizes may be, for example, 10 cm, 30 cm, and 60 cm. The different sizes may include different indicators 422 to indicate the relative size difference.

In some embodiments, the indicators 422 may be paired with weight estimates that are conveyed on the first portion 420 in addition to the indicators 422. This pairing of limb circumference values and weight estimates may allow for expedited medical treatment in case of emergencies.

Reference is now made to FIG. 4A, which illustrates a schematic view of the weight estimation device 104, according to some embodiments. In these embodiments, the weight estimation device 104 is separate from the strip 404.

The weight estimation device 104 may be any device capable of reading and/or receiving the data communicated by the second portion 430 of the strip 404. For example, the weight estimation device 104 may use, including, but not limited to, CCD, laser or camera-based reading technology, image reading technology, or any other method of receiving the limb circumference data conveyed by the second portion 430.

For example, as shown in FIG. 1A, the weight estimation device 104 is a barcode scanner. The indicators 422 on the first portion 420 are a barcode that may be scanned by the weight estimation device 104. Each mark in the indicators 422 may correspond to a different limb circumference measurement that may be read and converted by the weight estimation device 104 into a weight estimate. The weight estimation device 104 may be used to scan the second portion 430 to read the position of the indicator marker relative to the indicators 422. The weight estimation device 104 may compute the position of the indicator marker 436 and calculate the estimated weight of the patient.

In some embodiments, the weight estimation device 104 may include a storage region for storing one or more strips 404. This storage may improve the speed at which the user can apply new strips 404 to one or more patients.

Referring to FIG. 4B, shown therein is an exemplary schematic view of the weight estimation device 104 docked at a docking station 108. The docking station may be used to store and/or charge the weight estimation device 104. For example, the weight estimation device 104 may be wireless and powered by an onboard energy member, such as a battery. The docking station 108 may operate to charge the onboard energy member when the weight estimation device 104 is mounted to the docking station 108.

The docking station 108 may be placed, for example, on walls, medication carts, or hospital gurneys. The docking station 108 may act as storage for the strips 404, which may individually be pulled out of the docking station 108 for use around the patient's limb.

In some embodiments, the weight estimation device may be wired and may be stored on the patient's gurney.

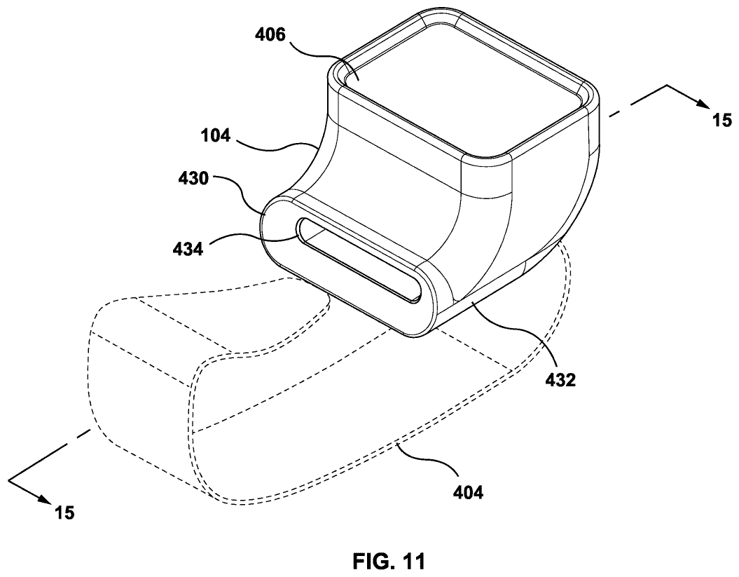

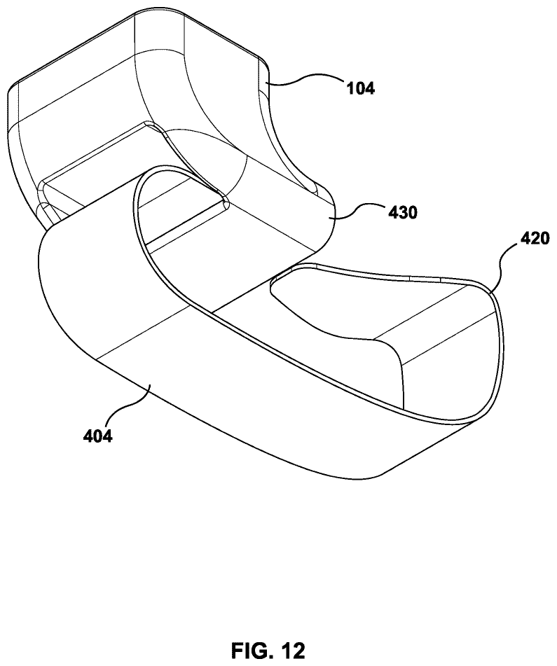

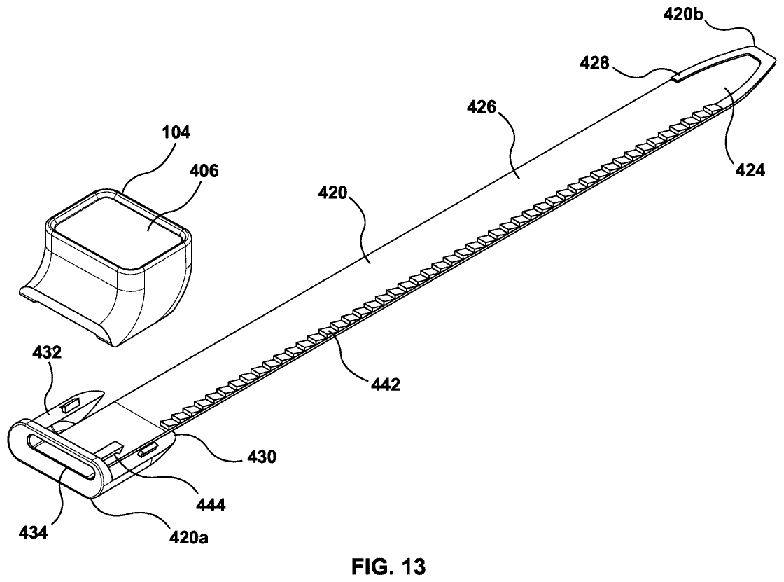

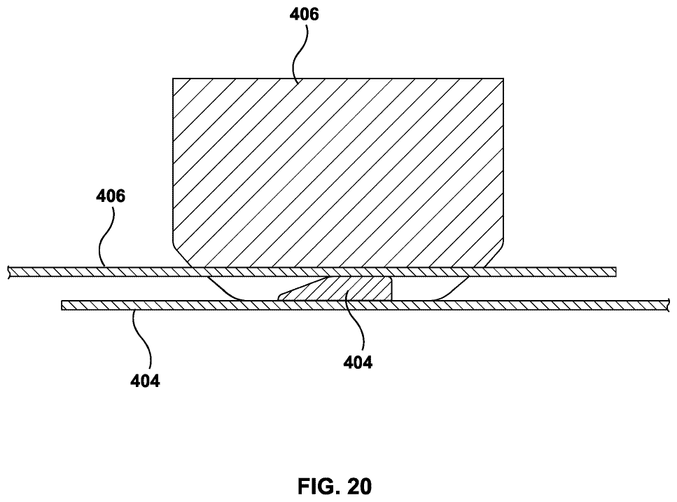

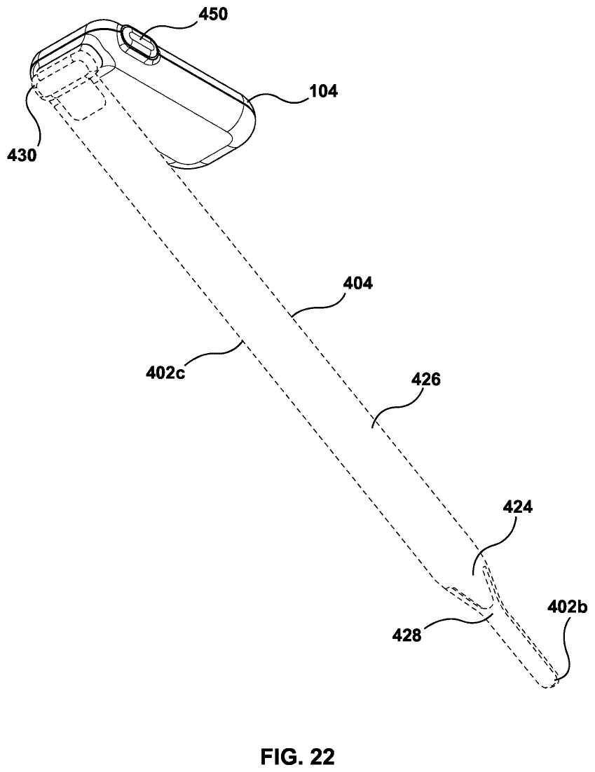

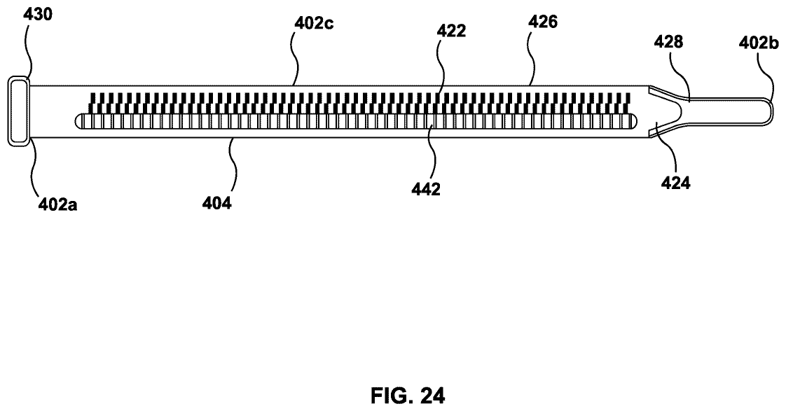

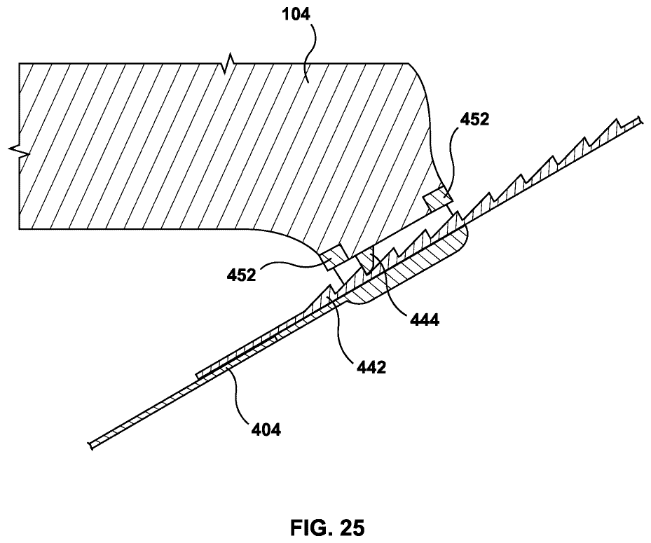

Reference is now made to FIGS. 11-25, which shows additional embodiments of system 200 for weight-based medication dose preparation. As will be appreciated by the skilled reader, the limb-based weight estimation devices described with reference to FIGS. 11-25 can be used with the weight-based medication dose preparation systems described herein.

As exemplified, in some embodiments, the limb-based weight estimation device 104 may be separate from the strip 404. As shown in FIGS. 11-25, the limb-based weight estimation device may be an encoder module 104 and the limb circumference measuring device 404 may be a strip.

An advantage of this design is that the encoder module 104 may be separatable from the strip 404. For example, when the strip 404 is in the mounted position and the encoder module 104 is coupled to the strip 404, the encoder module 104 may be spaced apart from the limb of the patient. Separating the encoder module 104 from the strip 404 may allow for simplified and/or reduced sanitization operations between patients. For example, in some embodiments, the encoder module 104 may not contact the limb of the patient during operation, limiting or negating the need for sanitization of the encoder module 104 between uses. The strip 404 may then be disposed of or sanitized after the use.

Referring to FIG. 11, shown therein is the encoder module 104 and the strip 404. The strip 404 is configured to be applied around the limb of the patient. The strip 404 is movable between an unmounted position and a mounted position on the limb of the patient. When in the mounted position, the strip engages around the patient's limb. When the strip 404 is mounted, the encoder module 104 may be used to estimate the weight of the patient based on the limb circumference indicated by the strip 404.

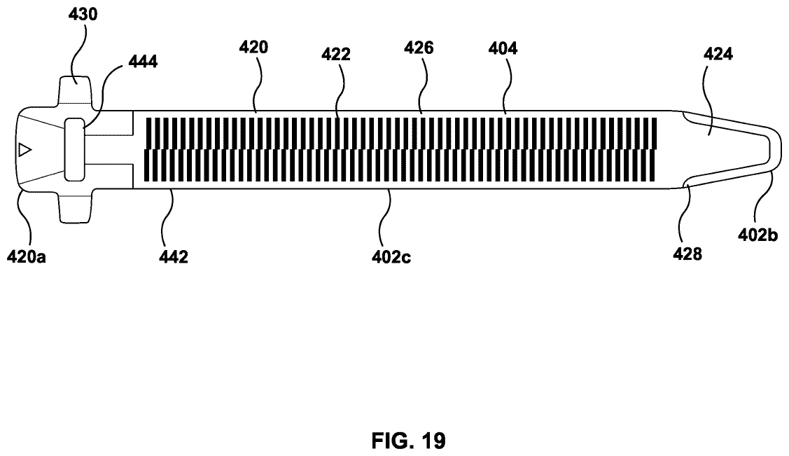

As exemplified in FIG. 17, the strip 404 has a first portion 420 including a plurality of indicators 422. The indicators may be of any type, including, but not limited to, one or more of optical, electronic, and/or magnetic. For example, the indicators 422 may be optical indicators that are incrementally spaced apart ticks on the first portion 420.

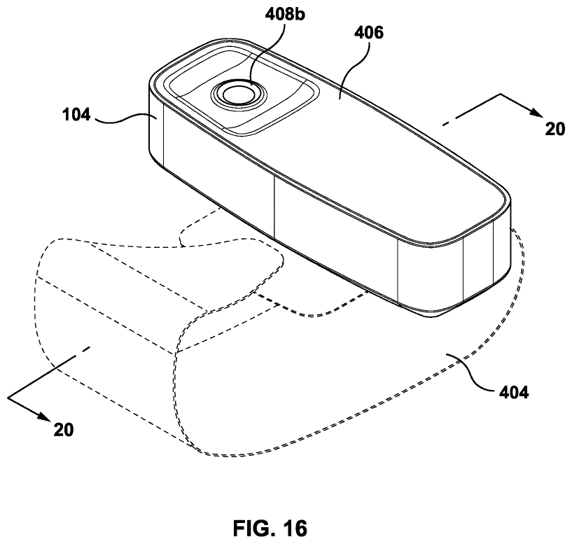

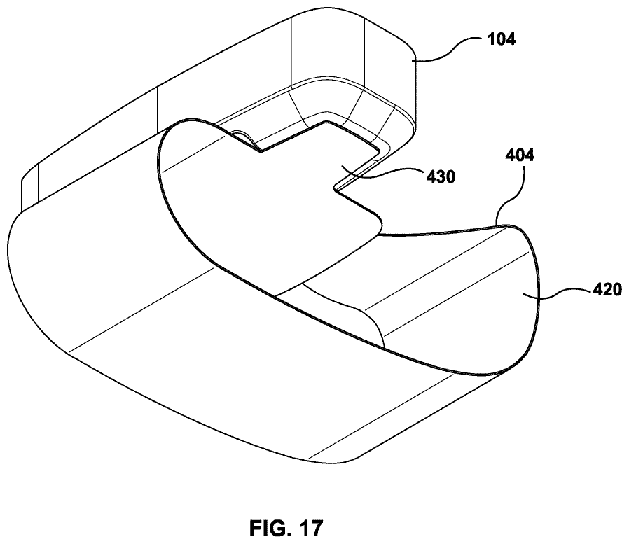

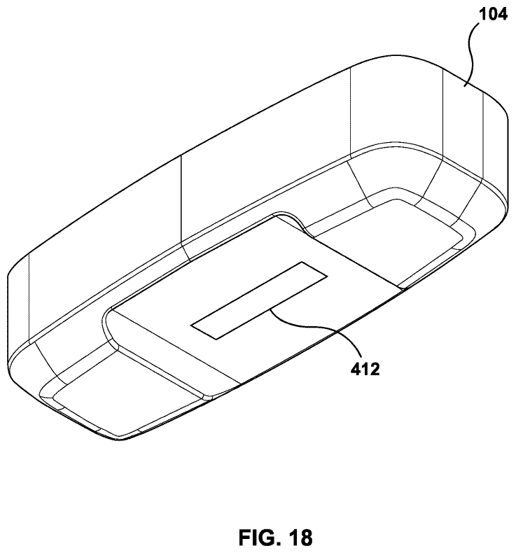

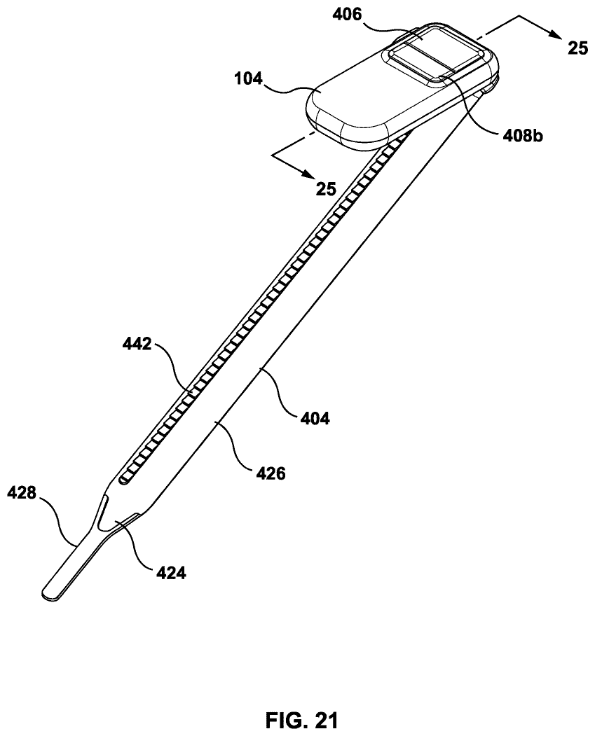

The strip 404 has a second portion 430 being configured to receive the encoder module 104. The encoder module 104 may be removably receivable by a housing 432 of the second portion 430. The encoder module 104 may be received by the second portion 430 in any way that enables the encoder module 104 to read the indicators 422. For example, the second portion 430 may include, but is not limited to, injection molding, snap-fit, adherence surface, hook and loop, magnetic, and/or combinations thereof. As exemplified in FIGS. 11 and 21, the second portion 430 may slidingly receive the encoder module 104. As exemplified in FIG. 16, the second portion 430 may adhesively receive the encoder module 104.

The second portion 430 may include an opening 434 for receiving the strip 404. As exemplified in FIG. 11, the opening 434 may be tapered to assist with the insertion of the second end 420b of the strip 404 to form the circumference around the limb of the patient.

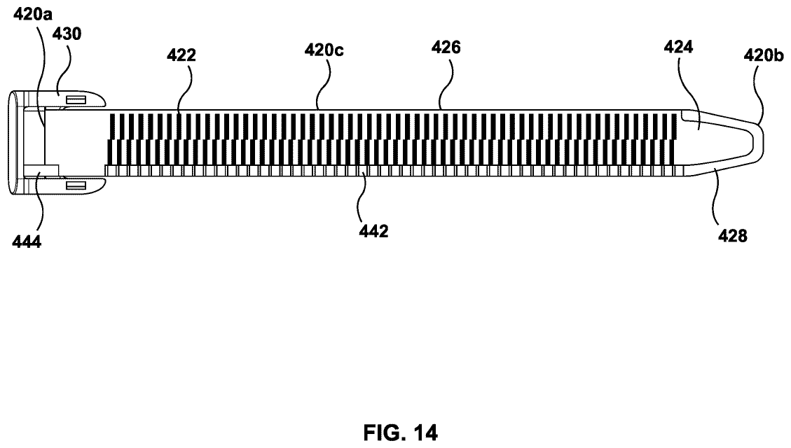

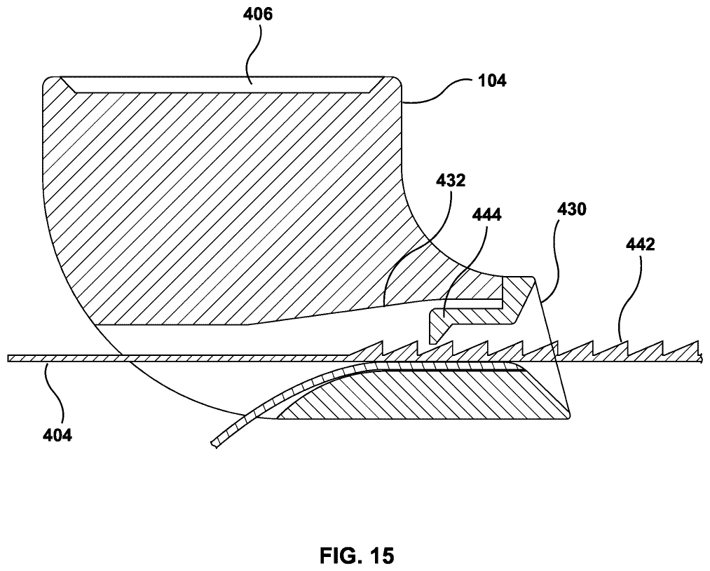

The strip 404 may vary in shape and/or size depending on the type of encoder module 104. For example, the relative width of the strip 404 may vary. Referring to FIG. 14, as shown, the strip has a first end 420a, a second end 420b, and a strip body 420c extending therebetween. As exemplified in FIG. 19, the first portion 420 of the strip 404 is narrower than the second portion 430 of the strip 404. The narrower first portion 420 may facilitate the ease of threading the second end 420b past the second portion 430 for mounting the strip 404 on the limb of the patient. The wider area proximate the second portion 430 may make it easier to couple the encoder module 104 to the strip 404. The wide area may also distribute pressure on the limb of the patient, relieving discomfort and/or improving the accuracy of the measurement by not pinching the limb. When the user is taking a measurement from the patient, the wide area may reduce slip along the limb of the patient, improving the accuracy of the measurement.

In some embodiments, the strip 404 may vary in other material properties, including, but not limited to, rigidity and manufacturing material. For example, as shown in FIG. 14, the strip body 420c has a semi-rigid section 424 proximate the second end 420b and a flexible section 426 between the semi-rigid section and the second portion 430. As shown, the semi-rigid section 424 is strengthened by the rigid member 428. The flexible section 426 may be made of a material that allows for the bending of the strip 404 around the limb of the patient, including, but not limited to, laminates. For example, the flexible section 426 may be made of Tyvek. The rigid member 428 may be laminated to the strip 404 to form the semi-rigid section 424. For example, the rigid member 428 may be made of hard plastic, nylon, and/or thermoplastics. The rigid member 428 may operate as a handle for the user to grip as they are mounting the strip 404 to the limb of the patient.

The encoder module 104 has an encoder 412 for monitoring the position of the first portion 420 of the strip 404 with respect to the second portion 430. The encoder 412 may be any type of device capable of reading the indicators 422. The encoder module 104 also has a device processor configured to determine, based on data received from the encoder, a circumference of the limb of the patient. The device processor is also configured to estimate a weight of the patient based on the circumference.

As shown in FIG. 2, the device processor of the encoder module 104 is capable of two-way communication with network 205. As such, the device processor may receive information from sources other than strip 404. In some embodiments, the device processor is further configured to use data received from the encoder as well as data received from external systems to estimate the weight of a patient. In some embodiments, the external systems may, for example, comprise Bolus Dose Preparation System 110 and/or hospital records. In some embodiments, the processor is further configured to determine and/or receive other information, such as other patient data (e.g., weight, gender, medical condition), to estimate a weight of the patient based on the circumference and the other patient data.

The weight estimation device may include a tensioning mechanism 440 that allows for a variable length of the circumference of the mounted strip 404 to decrease from a first length to a second length with an applied tensile force. For example, when at the first length, the user may tighten the first portion 420 by pulling on the second end 420b of the first portion 420 to reduce the circumference. The tensioning mechanism 440 may provide a one way pull mechanism such that the length does not increase once being tightened on the limb of the patient. This tensioning mechanism 440 may improve the accuracy of the limb circumference measurement by preventing backslide of the first portion 420 due to, e.g., movement of the user and/or flexing of the muscle of the limbs.

The tensioning mechanism 440 may include a sliding member 442 and a resistance member 444. The sliding member 442 may be configured to move past the resistance member 444 in a first direction to decrease the circumference of the mounted strip 404 and may be resisted from moving a second direction opposite the first direction by the resistance member 444.

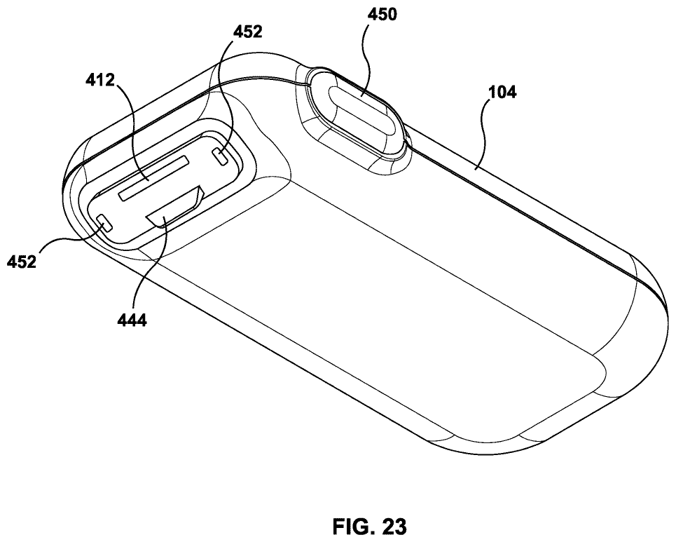

The sliding member 442 may be positioned on the first portion 420 of the strip 404. The resistance member 444 may be positioned on the strip 404 and/or on the encoder module 104. As exemplified in FIGS. 14 and 19, the resistance member 444 is positioned on the strip 404 and as exemplified in FIG. 23, the resistance member is positioned on the encoder module 104.

The resistance member 444 may be any material or mechanism that resists the movement of the sliding member 442 as the strip 404 is being mounted to the limb of the patient. For example, the resistance member 444 may cause resistance due to friction. As shown in FIG. 19, the resistance member 444 is a closed-cell foam. When the sliding member 442 is slid over the foam 444 and is pinched between the foam 444 and the encoder module 104, the sliding member 442 may be resisted from moving backwards.

In some embodiments, the tensioning mechanism 440 may include, for example, a ratcheting system such as a clip and teeth mechanism. As exemplified in FIG. 15, the clip and teeth mechanism may include a pawl 444 that can ratchet the teeth 442 on the first portion 420 such that the strip 404 can be tightened around the limb of the patient. Accordingly, the variable length of the strip 404 may be limited to decreasing in length once the tensioning mechanism 440 is engaged.

In some embodiments, the resistance member 444 may be removeable from the weight estimation device. For example, referring to FIG. 23, the clip 444 may be a silicone tongue that is removeable from the encoder module 104. As shown, the encoder module 104 includes an eject button 450 for disengaging the encoder module 104 from the resistance member 444 and the strip 404 using ejector pins 452. An advantage of this design is that the portion of the encoder module 104 that contacts the strip 404 may be ejected from the encoder module 104, reducing the risk of spreading contaminates from the strip 404 to the encoder module 104. This reduction in contamination may simplify or negate the need to sanitize the encoder module 104 between subsequent uses. The eject button 450 may also serve the function of unmounting the strip 404 to simplify its removal after the weight measurement has been taken.

In some embodiments, the tensioning mechanism 440 may allow for incremental decreasing of the length of the circumference. For example, the tensioning mechanism 440 may allow for 1 mm incremental movement of the strip 404. This incremental movement may improve the accuracy of the measured limb circumference.

The strip 404 and tensioning mechanism 440 may be operated manually, automatically, or both. For example, the user may manually pull on the second end 420b of the first portion 420 to tighten the strip 404 and engage the tensioning mechanism 440. In some embodiments, the tensioning mechanism 440 may automatically operate the pawl 444 to tighten the circumference to the second length. For example, the tensioning mechanism 440 may include a motor that activates the pawl 444. In some embodiments, the strip 404 may be tightened by, for example, an air pump paired with a valve.

To assist with accurate and consistent measurements, the tensioning mechanism 440 may limit the applied force exerted on the limb of the user. For example, the tensioning mechanism 440 may include teeth 442 that are frangible at specific tensile force thresholds so as to yield the same pressure around the patient's limb when mounted. In some embodiments, the pawl 444 of the tensioning mechanism 440 may be fitted with one or several force transducers to alert the user when the proper pressure has been obtained around the patient's limb. In some embodiments, the strip 404 may include a motor for automatic application of tensile force and force transducers that automatically alert the system 200 when the proper pressure has been reached. These safety mechanisms may also reduce the likelihood of injury to the patient by overtightening the strip 404.

In some embodiments, the weight estimation system 200 may include a plurality of strips 404 with a plurality of strip types. Each strip type may be different in size for e.g., differently aged patients. The device processor may be configured to determine the type of strip 404 and use the type of strip to estimate the weight of the patient. For example, the algorithm used to determine the weight of the patient may be modified by the strip type. The type of strip may be indicated by at least one of visual, magnetic, and/or electronic indicators. For example, the strips 404 may be colour-coded to indicate different lengths of strip 404. In some embodiments, the encoder module 104 may be configured to determine the type of strip 404 and may provide an indication to the user of which strip type is being used by the algorithm, thereby allowing the user to verify the appropriateness of the algorithm being used for the measurement.

Some embodiments include a wall-mounted or cart-mounted or hub for holding and/or dispensing the strips. The hub is designed to guide the user in selecting the right strip and to ensure adequate fitting between the age-dependent strip and the encoder module (helps the user snap-fit the two pieces together correctly). In some embodiments of this hub, the placement of the encoder in its hub will automatically discard and/or sanitize the strips. In some embodiments, the hub will charge the encoder module. In yet other embodiments, the hub will warn the user of any issues with the encoder module (e.g., not connected to medication calculation software, not calibrated, etc.).

Reference is now made to FIG. 7, which shows a simplified block diagram of hardware for an example embodiment of a weight estimation device 104 and a computer terminal 106.

As shown, the weight estimation device 104 can include a processor 502a connected, via a data bus, to one or more of memory 504a, communication interface 506a, one or more encoder unit(s) 508a, one or more sensors 510a, a display interface 512a and one or more input interfaces 514a.

Processor 502a is a computer processor, such as a general-purpose microprocessor. In some other cases, processor 502a may be a field programmable gate array, application specific integrated circuit, microcontroller, or other suitable computer processor. In some cases, processor 502a may in fact comprise multiple processors.

Processor 502a is coupled, via a computer data bus, to memory 504a. Memory 504a may include both volatile and non-volatile memory. Non-volatile memory stores computer programs consisting of computer-executable instructions, which may be loaded into volatile memory for execution by processor 502a as needed. It will be understood by those of skill in the art that references herein to the weight estimation device 104 as carrying out a function or acting in a particular way imply that processor 502a is executing instructions (e.g., a software program) stored in memory 504a and possibly transmitting or receiving inputs and outputs via one or more interface. Memory 504a may also store data input to, or output from, processor 502a in the course of executing the computer-executable instructions. In at least some embodiments, memory 504a may store a weight estimation software 516a which can convert circumference data into estimated weight.

The docking station 108 may act as additional data storing and/or memory capacity to supplement the weight estimation device 104 or store weight-based patient data locally.

Communication interface 506a is one or more data network interface, such as an IEEE 802.3 or IEEE 802.11 interface, for communication over a network. In some cases, communication interface 506a may be configured for Bluetooth® communication.

Primary measurement sensor(s) 508a may be analogous to encoder unit(s) 412. As discussed above, encoder unit(s) 508a may comprise one or more linear and/or rotary encoders for measuring limb circumference using strip 404. In other cases, primary measurement sensor(s) 508a may comprise various types of motion sensors for tracking and/or recording movement of the strip 404 into the mounted position, i.e., as it is being applied around the patient's limb.

Secondary sensor(s) 510a may include, as also stated previously, pressure, force, or tactile sensors for determining correct application of the weight measuring device 104 in the mounted position. Secondary sensors 510a can also include sensors for measuring blood pressures, brachial pulse, bone density, body temperature, and/or oxygen saturation, as well as broadly any other sensors for monitoring patient biometric data.

Display interface 512a may be analogous to display interface 406, which is a suitable display for outputting information and data as needed (e.g., weight estimates). In some cases, display interface 512a comprises a liquid crystal display (LCD).

Input interface 514a may comprise any suitable interface for receiving user inputs, and may include buttons described herein.

Similar to weight estimation device 104, computer terminal 106 may also include processor 502b which is connected, via a data bus, to memory 504b, communication interface 506b, display interface 508b and input interface 510b. The components of the computer terminal 106 may be analogous in structure and function to the equivalent components as described in relation to the weight estimation device 104. As discussed, memory 504b may store the medication dose preparation software 512b.

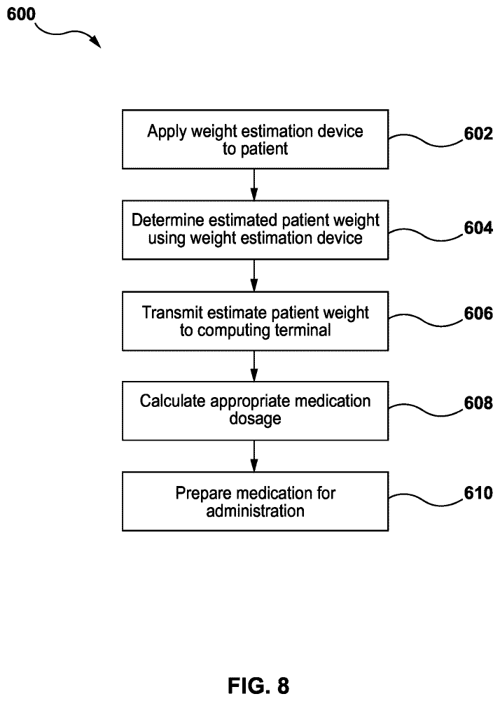

Reference is now made to FIG. 8, which shows a process flow for an example embodiment of a method 600 for medication dose preparation.

At 602, the weight estimation device 104 is applied to the patient's limb.

At 604, the weight estimation device 104 can estimate the patient's weight based on the limb circumference. For example, this may be performed using a circumference-based weight prediction algorithm. In various cases, the patient weight may be estimated and/or monitored in real-time or near real-time.

At 606, the patient's estimated weight may be transmitted to the external computing device 106, which host the medication dose software 512b. In other cases, the weight estimation device 104 can simply transmit the measured circumference data. In still other cases, weight estimation device 104 can transmit various sensor data associated with sensors located in the weight estimation device 104.

At 608, the medication dose software 512b—operating on the computing terminal 106—may calculate the appropriate medication dosage parameters. The dosage parameters may comprise various quantities associated with the medication dose to be administered to the patient based on the medication type, and one or more of the (i) the patient weight and (ii) other patient-specific data (i.e., patient age, gender, medical history, etc.). By way of non-limiting examples, the dosage parameters can include a suggest dosage of the medication, total dosage to administer of the medication, stock concentration of the medication, volume of medication to draw and volume of diluent. In various cases, the patient's estimated weight is determined based on data received from the weight estimation device 104. Further, the patient-specific data may be entered by a user into the medication dose software, or otherwise received externally, i.e., from server 210a.

At 610, the medication dose is prepared based on the determined medication dose parameters. In some cases, the medication dose preparation is performed manually, i.e., by a health care practitioner. In at least some embodiments, the medication dose software may provide guiding instructions to assist the practitioner in preparing the medication dose, as well as prioritizing between several types of medication to be administered. In other cases, act 610 may also be performed automatically. For example, the medication dose software 512b may be operable to control an automated bolus dose preparation system 110 (FIG. 1B).

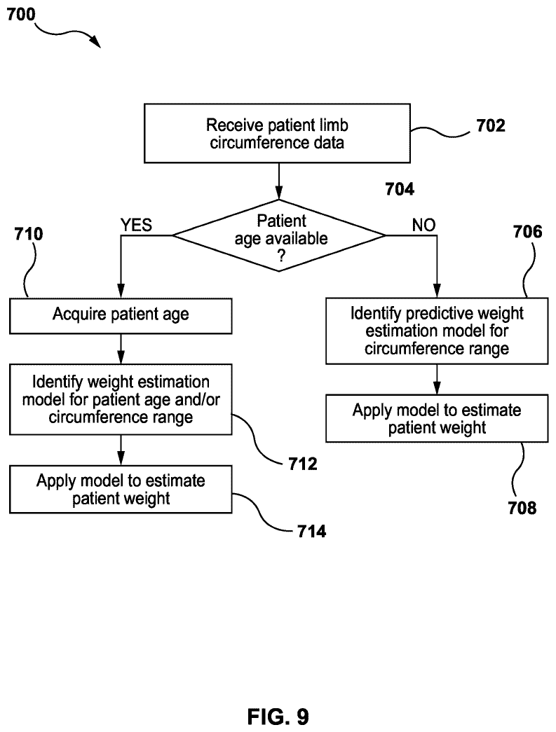

Reference is now made to FIG. 9, which shows a process flow for an example embodiment of a method 700 for automated weight estimation (i.e., act 604 in method 600). Method 700 can be performed using one or more of the processor 502a of the weight estimation device 104 and/or the processor 502b of the computer terminal 106.

At 702, the weight estimation software 516a—operating on either the weight estimation device 104 or computer terminal 106—may receive patient limb circumference data. For example, the limb circumference data may be generated by the weight estimation device 104 applied around the patient's limb, as previously described.

At 704, a determination is made as to whether the patient's age is available. As provided herein, the patient's age may be used to enhance the predictive accuracy of the weight estimation model.

If the patient's age is not available, then method 700 may proceed to acts 706 and 708. At 706, the weight estimation software 516a can identify a suitable predictive weight estimation model based on the measured limb circumference. In at least one embodiment, different predictive models may be selected for different limb circumference ranges. For example, a first predictive model can be used for limb circumferences in a first range (i.e., below 15 centimeters (cm)), a second predictive model can be used for limb circumferences in a second range (i.e., between 15 cm and 30 cm), and a third predictive model can be used for limb circumferences in a third range (i.e., above 30 cm). To this end, it has been appreciated that, to increase weight prediction accuracy-rather than relying on a single predictive model, a combination of predictive models can be used to accommodate for variations in limb circumferences. In particular, each predictive model may comprise a combination of linear and non-linear functions that relate limb circumference to patient weight.

At 708, the limb circumference data may be applied to the selected weight estimation model to generate an estimate of the patient's weight.

Returning to act 704, if the patient's age is available—at 710, the weight estimation software 516a can acquire the patient's age. In some cases, act 706 may be performed prior to act 704 such that if the software is unable to acquire the patient's age, the method may automatically revert to acts 706 and 708.

In at least one embodiment, at 710, the patient's age is manually acquired. For instance, the input interface 514a to the weight estimation device 104—or the input interface 510 to the computer terminal 106—may allow a user (i.e., medical practitioner) to manually input the patient's age.

In other embodiments, the patient's age may be acquired through more automated methods. For example, a scanning system may couple to either the weight estimation device 104 and/or computer terminal 106. The scanning system can comprise, for example, a camera or a barcode reader. The scanning system may be employed to scan (or read) a scannable indicium associated with the patient. For example, this may be a scannable indicium, i.e., a barcode or QR code, located on a patient identification wristband.

In at least one case, the scanning system may scan the indicia to automatically acquire the patient's age. In other cases, the scanning system may scan the indicia to retrieve secondary information, such as a patient ID number. The weight estimation software 516a can then cross-reference the acquired patient ID to a database storing patient-specific data, i.e., including the patient's age. The cross-referenced database may be a local database stored on the weight estimation device memory 504a and/or computing terminal memory 512b. In other cases, the database may be externally hosted on one or more of servers 210a, 210b. In still other cases, the patient ID—if known—may also be manually input by a user into the input interfaces 514a, 514b.

At 712, the weight estimation software 516a may identify a suitable weight estimation model based on both the measured limb circumference and the patient's age.

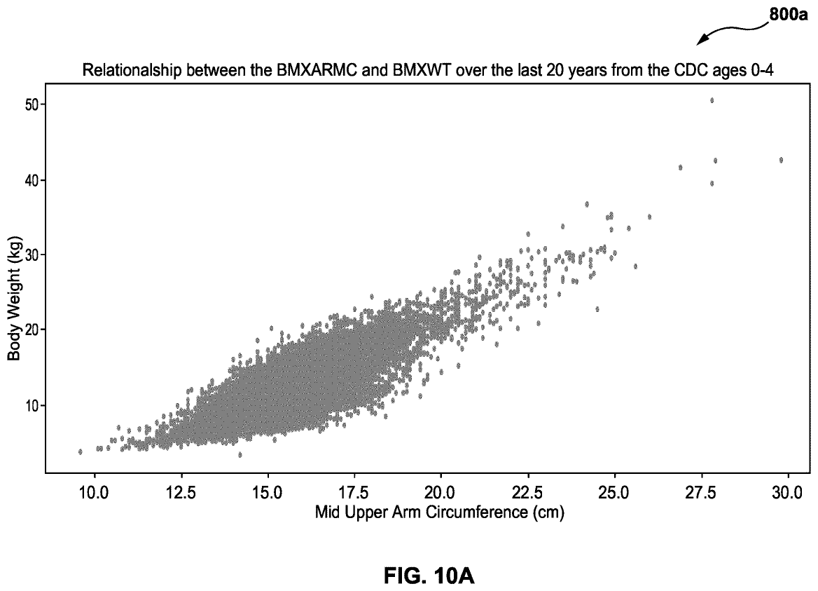

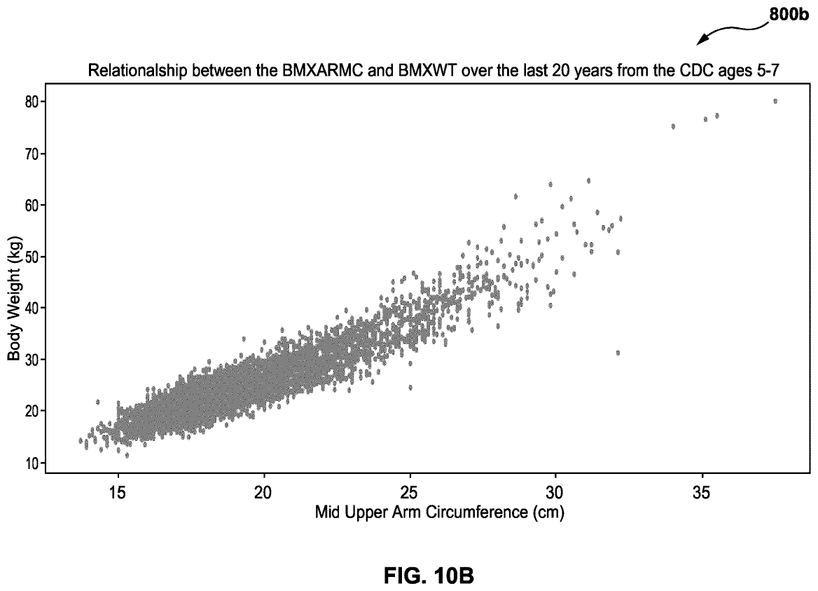

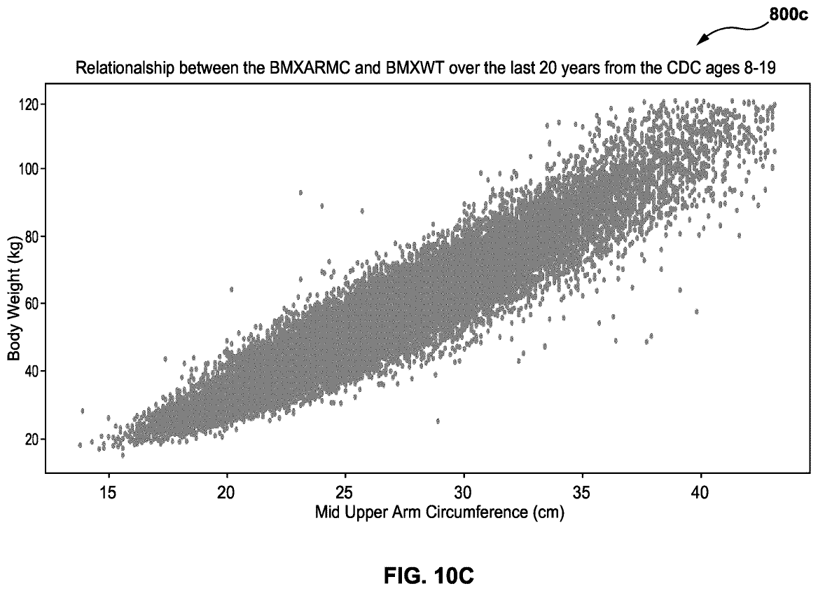

In at least one embodiment, different estimation models may be used based on the patient's age range. For example, FIGS. 10A and 10B, and 10C show plots 800a, 800b, and 800c, respectively, illustrating the correlation between arm circumference (centimeters) and body weight (kilograms) for subjects within the age ranges of 0 to 4 years old (FIG. 10A), 5 to 7 years old (FIG. 10B) and 8 to 19 years old (FIG. 10C). Equation (1) shows a linear regression model generated based on plot 800a, while Equations (2) and (3) show linear regression models generated based on plot 800b and 800c, respectively.

$$Weight = 1.25991537 * ( Arm Circumference ) + 1.66472123 * ( Age ) - \text{.10} \text{.054796602458609} \qquad ( 1 )$$

$$Weight = 2.0528604 * ( Arm Circumference ) + 1.39655423 * ( Age ) - 23.24261052651997 \qquad ( 2 )$$

$$Weight = 3.19321569 * ( Arm Circumference ) + 1.01703969 * ( Age ) - 42.74004008992316 \qquad ( 3 )$$

Equation (1) yields an average predictive error of approximately 8.04%, and an R² value of approximately 0.90. Equation (2) yields an average predictive error of approximately 6.73%, and an R² value of approximately 0.89 . . . . Equation (3) yields an average predictive error of approximately 7.84%, and an R² value of approximately 0.92. Tables 1, 2 and 3, below, illustrate the percentage agreement between the expected and predicted data using Equation (1) (Table 1), Equation (2) (Table 2), and Equation (3) (Table 3).

TABLE 1
Percentage in agreement within

| 10% | 20% | 30% | 40% | 50% | 60% | 70% | 80% | 90% | 100% | >100% |
|---|---|---|---|---|---|---|---|---|---|---|
| 70.4% | 93.8% | 98.8% | 99.7% | 100% | 100% | 100% | 100% | 100% | 100% | 100% |

TABLE 2
Percentage in agreement within

| 10% | 20% | 30% | 40% | 50% | 60% | 70% | 80% | 90% | 100% | >100% |
|---|---|---|---|---|---|---|---|---|---|---|
| 77.1% | 97.9% | 99.8% | 99.9% | 99.9% | 100% | 100% | 100% | 100% | 100% | 100% |

TABLE 3
Percentage in agreement within

| 10% | 20% | 30% | 40% | 50% | 60% | 70% | 80% | 90% | 100% | >100% |
|---|---|---|---|---|---|---|---|---|---|---|
| 69.4% | 95.9% | 99.6% | 99.9% | 99.9% | 99.9% | 100% | 100% | 100% | 100% | 100% |

Accordingly, a two-feature predictive model (i.e., based on age and weight) is expected to generate higher accuracy weight predictions.

In at least one embodiment, within each age group, separate predictive models are provided for different circumference ranges, as explained with reference to act 706. For example, for a given age group: a first age-specific predictive model can be used for limb circumferences in a first range (i.e., below 15 centimeters (cm)), a second age-specific predictive model can be used for limb circumference in a second range (i.e., between 15 cm and 30 cm), and a third age-specific predictive model can be used for limb circumferences in a third range (i.e., above 30 cm). Accordingly, at 712, identifying the correct weight estimation model may involve: (a) initially, identifying the appropriate age range; and (b) subsequently identifying the appropriate circumference range.

At 714, the selected predictive model is applied to estimate patient weight.

In some cases, the weight estimation device 104 may be used to measure circumferences for different types of limbs (i.e., mid upper arm, thigh, calf etc.). In these cases, the weight estimation device 104 may enable users to select the particular limb type to measure (i.e., via the input interface 514a). At 702, the weight estimation software 516a may receive the limb circumference data, as well as an indicator of the limb type associated with the circumference data. At 706 or 712, the weight estimation software 516a may identify the set of predictive models that are applicable to that limb type (i.e., based on limb circumference range and/or patient age). Accordingly, the predicted weight may be tailored for the measured limb.

In some embodiments, the weight estimation software may validate that a patient's weight is within 5th and 95th population percentiles and may further provide an indication (such as an alarm) to the user of a potential error when outside of these ranges. The weight validation may be performed using weight for length and/or weight for age World Health Organization (WHO) data or using weight for length and/or weight for age Center for Disease Control (CDC) data. In either case, the patient's weight may be compared against the WHO or CDC standards for the same length (height) or age, and a percentile calculation is performed.

The present invention has been described here by way of example only, while numerous specific details are set forth herein in order to provide a thorough understanding of the exemplary embodiments described herein. However, it will be understood by those of ordinary skill in the art that these embodiments may, in some cases, be practiced without these specific details. In other instances, well-known methods, procedures and components have not been described in detail so as not to obscure the description of the embodiments. Various modification and variations may be made to these exemplary embodiments without departing from the spirit and scope of the invention, which is limited only by the appended claims.

1. A weight estimation device comprising:
a disposable strip configured to be applied around a limb of a patient, the strip having a first portion and a second portion, the first portion including a plurality of indicators and the second portion being configured to receive an encoder module for monitoring the position of the first portion of the strip with respect to the second portion of the strip using the indicators,
wherein the disposable strip is movable between an unmounted position and a mounted position and when in the mounted position, the strip engages around the patient's limb, and
wherein the disposable strip is configured such that, when in the mounted position and the encoder module is coupled to the second portion, the encoder module is spaced apart from the limb of the patient.

2. (canceled)

3. (canceled)

4. The weight estimation device of claim 1, further comprising a tensioning mechanism having a sliding member and a resistance member.

5. The weight estimation device of claim 4, wherein the resistance member causes resistance due to friction.

6. (canceled)

7. The weight estimation device of claim 4, wherein the resistance member includes a ratchet system having a clip and teeth mechanism.

8. The weight estimation device of claim 7, wherein the teeth are positioned on the sliding member.

9. The weight estimation device of claim 7, wherein the clip is positioned on one of the disposable strip and the encoder module.

10. The weight estimation device of claim 4, wherein the sliding member is positioned on the disposable strip and the resistance member is positioned on one of the disposable strip and the encoder module.

11. The weight estimation device of claim 9, wherein the clip is positioned on the encoder module and is removeable from the encoder module.

12. The weight estimation device of claim 1, wherein the disposable strip is configured to removably receive the encoder module in the second portion of the disposable strip.

13. The weight estimation device of claim 12, wherein the disposable strip is configured to slidingly receive the encoder module in the second portion of the disposable strip.

14. The weight estimation device of claim 12, wherein the disposable strip is configured to adhesively receive the encoder module in the second portion of the disposable strip.

15. (canceled)

16. The weight estimation device of claim 1, wherein:
the disposable strip has a first end and a second end with a disposable strip body extending therebetween, the first end being proximate the second portion, and
the disposable strip body has a semi-rigid section proximate the second end and a flexible section between the semi-rigid section and the second portion.

17-37. (canceled)

38. A disposable strip configured to be applied around a limb of a patient, the strip being movable between an unmounted position and a mounted position, and when in the mounted position, the disposable strip forms a variable circumference that engages around the limb, the disposable strip having:
a first portion and a second portion, the first portion including a plurality of indicators and the second portion being configured to receive an encoder module; and
a tensioning mechanism for varying the circumference, the tensioning mechanism having a sliding member and a resistance member,
wherein the sliding member is operable to move past the resistance member in a first direction to decrease the circumference and is resisted from moving in a second direction opposite the first direction by the resistance member.

39. The disposable strip of claim 38, wherein the plurality of indicators are at least one of optical indicators and magnetic indicators.

40. (canceled)

41. The disposable strip of claim 38, wherein the resistance member causes resistance due to friction.

42. (canceled)

43. The disposable strip of claim 38, wherein the resistance member includes a ratchet system having a clip and teeth mechanism.

44. The disposable strip of claim 38, wherein the disposable strip is configured to receive the encoder module in the second portion of the disposable strip.

45. The disposable strip of claim 44, wherein the disposable strip is configured to slidingly receive the encoder module in the second portion of the disposable strip.

46. The disposable strip of claim 44, wherein the disposable strip is configured to adhesively receive the encoder module in the second portion of the disposable strip.

47. (canceled)

48. The disposable strip of claim 38, wherein the disposable strip further comprises a first end and a second end with a disposable strip body extending therebetween, the first end being proximate the second portion, and
the disposable strip body has a semi-rigid section proximate the second end and a flexible section between the semi-rigid section and the second portion.

49-80. (canceled)
